(19) United States
(12) Patent Application Publication
(10) Pub. No.: US 2024/0415780 A1
(43) Pub. Date: Dec. 19, 2024

(54) Neuronal diencephalon stem cells and exosomes thereof for the treatment and prevention of diseases

(71) Applicant: Florica Therapeutics, Inc., San Francisco, CA (US)

(72) Inventor: Christine Ichim, Livermore, CA (US)

(21) Appl. No.: 17/913,406

(22) PCT Filed: Mar. 19, 2021
(86) PCT No.: PCT/US21/23320

§ 371 Date: Sep. 21, 2022

Related U.S. Application Data
(60) Provisional application No. 62/992,907, filed on Mar. 21, 2020.
(60) Provisional application No. 62/994,838, filed on Mar. 25, 2020.
(60) Provisional application No. 62/994,843, filed on Mar. 26, 2020.
(60) Provisional application No. 62/994,863, filed on Mar. 26, 2020.

(51) Int. Cl. A61K 9/50; A61K 35/30; A61K 38/18; A61K 38/27; A61K 38/29; C12N 5/0793; C12Q 1/6881; G01N 33/50

(52) U.S. Cl. CPC A61K 9/5068; A61K 35/30; A61K 38/1858; A61K 38/1875; A61K 38/27; A61K 38/29; C12N 5/0619; C12Q 1/6881; G01N 33/5005; C12Q 2600/158

(57) ABSTRACT

Methods of preventing, treating, managing or controlling diseases in humans by delivery of compositions comprising neuronal stem cells and exosomes of di encephalon lineage are disclosed. Also provided are pharmaceutical compositions that comprise neuronal stem cells or exosomes of diencephalon lineage, for the treatment and prevention of disorders associated with the neuroendocrine system, the control of behavioral and physiological processes, and therapy for viral and other diseases.

### CROSS-REFERENCE TO RELATED APPLICATIONS

The present application claims priority to U.S. Provisional Application No. 62/994,863, filed Mar. 26, 2020, U.S. Provisional Application No. 62/994,838, filed Mar. 25, 2020, U.S. Provisional Application No. 62/994,843, filed Mar. 26, 2020, and U.S. Provisional Application No. 62/992,907, filed Mar. 21, 2020, the contents of which are incorporated by reference in their entirety.

### FIELD OF INVENTION

Methods of preventing, treating, managing or controlling diseases in humans by delivery of compositions comprising neuronal stem cells and exosomes of diencephalon lineage are disclosed. Also provided are pharmaceutical compositions that comprise neuronal stem cells or exosomes of diencephalon lineage, for the treatment and prevention of disorders associated with the neuroendocrine system, the control of behavioral and physiological processes, and therapy for viral and other diseases.

### BACKGROUND OF INVENTION

The potent and safe delivery of therapeutics across the blood-brain barrier is a major challenge for treating diseases whose pathophysiology is related to the brain. Moreover, many drugs and therapeutics may not technically or safely be delivered to whole organisms because of limitations in their bio-distribution, stability or toxicity.

RNA, recombinant RNA-based, siRNA, miRNA, lncRNA, antisense oligonucleotide and other oligonucleotide-based technologies, including methods used to regulate gene expression in vitro, have the potential to treat a large number of diseases. In practice however, the use of nucleotide-based therapeutics is hampered by ineffective drug targeted delivery.

Thus, although the brain is the epicenter of a number of serious neurological and physiological disorders and cancer-based diseases, the difficulty in transferring active molecules across the blood-brain barrier and drug toxicity are major causes in lowering successful treatment rates.

Therefore, there is a clear need for development of novel methods of delivering therapeutics across the blood-brain barrier, which would enable treatment of numerous life-threatening diseases.

The hypothalamus is a small region of the brain, located at the base of the brain, near the pituitary gland, and known to play a crucial role in various functions, including hormone release, body temperature regulation, daily physiological cycle maintenance, appetite control, sexual behavior and emotional responses.

The anterior region of the hypothalamus, the supraoptic region, is involved in the secretion of hormones, such as corticotropin-releasing hormone (CRH), thyrotropin-releasing hormone (TRH), gonadotropin-releasing hormone (GnRH), oxytocin, vasopressin and somatostatin, and in maintaining circadian rhythms. The middle region of the hypothalamus, the tuberal region, regulates appetite and the growth hormone-releasing hormone (GHRH). The posterior area of the hypothalamus, the mammillary region, regulates body temperature.

Because of their strategic location, hypothalamic neurons can sense both neural and physiological signals, and respond by releasing neurotransmitters and neuromodulators into the brain. Human hypothalamic neuron dysfunction has been related to a variety of diseases, such as obesity, hypertension, and mood and sleep disorders. Therefore, the production of human hypothalamic neurons and exosomes thereof in vitro can be very helpful in the understanding of hypothalamic neuron dysfunction-related diseases in humans and treatment of these diseases by targeted delivery.

However, the current technologies for creating, manufacturing and scaling up the production of human hypothalamic stem cells and exosomes thereof suffer from several drawbacks.

Hypothalamic extracellular vesicles and exosomes are produced from stem progenitor and mature cells. The production of extracellular vesicles or exosomes from human hypothalamus cells requires a human hypothalamic cell source. However, hypothalamic cells are difficult to obtain, and it is exceedingly difficult to source human hypothalamic stem cells. This problem is due, in part, to the fact that the hypothalamus constitutes only 0.3% of the brain and obtaining human hypothalamic cell sources requires access to cadavers. Obtaining human hypothalamic stem cells is even more challenging, as the process requires the use of a child cadaver.

Methods aimed at devising culture conditions that stimulate the differentiation of pluripotent cells into neurons of hypothalamus lineage are not reproducible across pluripotent cell lines and give rise to mature hypothalamus cells of low purity. The identification of the specific stage at which hypothalamus stem cells are formed along this differentiation pathway is particularly challenging. Furthermore, commercial large-scale manufacture of these cells has not yet been developed, and the conditions that allow for the isolation of extracellular vesicles or exosomes from human hypothalamic stem and progenitor cells derived from pluripotent cells have not been defined.

Alternative economically viable solutions for the creation, manufacturing and mass production of human hypothalamic stem and/or progenitor cells and extracellular vesicles or exosomes of such cells are therefore needed.

### SUMMARY OF THE INVENTION

The present application presents a solution to the aforementioned challenges by providing therapies based on drug-targeted delivery that make use of human hypothalamus-derived exosomes. The disclosed human hypothalamus stem cells and exosomes are produced by reliable, cost-effective and easily scalable methods that allow isolation and purification of human hypothalamus stem cells and exosomes during induced pluripotent cell in vitro differentiation, and are formulated as pharmaceutical formulations or as loaded vectors for targeted delivery of drugs or active compounds for the treatment and prevention of a variety of diseases, including disorders associated with the neuroendocrine system, immunological and viral diseases, and the control of behavioral and physiological processes.

Thus, in some embodiments, provided herein is a method for preventing, treating, managing or controlling a disease or disorder associated with hypothalamus malfunction in a subject in need thereof. The disclosed method comprises administering to the subject a pharmaceutical composition comprising a therapeutically effective amount of purified human hypothalamus exosomes, that are engineered to express one or more foreign molecules, or are loaded with a biologically active compound.

Thus, in some embodiments, provided herein is a method for identifying, isolating and purifying human hypothalamus stem cells at an intermediate transitory stage during in vitro induced pluripotent stem cell differentiation, which corresponds to human hypothalamus stem cell formation. The disclosed method comprises: (i) culturing induced pluripotent stem cell in culture media and enabling reproducible differentiation of induced pluripotent stem cells into cells of ventral diencephalon hypothalamic cell lineage; (ii) identifying hypothalamic cells between day 0 and day 15 of cell culture that display hypothalamus markers and neuronal stem cell markers; (iii) isolating hypothalamus stem cells at hypothalamus marker and neuronal stem cell marker maximal expression; and (iv) purifying isolated human hypothalamus stem cells.

Neuronal stem cell markers that may be used to detect human hypothalamus stem cell formation include, but are not limited to, one or more of Sox2⁺, Bmi-1⁺, nestin⁺, Musashi1⁺ Cxcr4⁺.

Hypothalamus markers that may be used to detect human hypothalamus stem cell formation include, but are not limited to, one or more of NK2 homeobox 1 (Nkx2.1) and homeobox protein orthopedia.

In some embodiments, the purified human hypothalamus stem cells obtained by the disclosed method are Rax^{hi}, Sox1^{lo}, Sox2⁺ and Bmi-1⁺.

In some embodiments, hypothalamus marker and neuronal stem cell marker maximal expression is between day 7 and day 15 of cell culture.

In some embodiments, the purified human hypothalamus stem cells obtained by the disclosed method are tanycytes.

In some embodiments, the disclosed method may further comprise analyzing the purified human hypothalamus stem cells for immunophenotype, neurosphere formation and ability to give rise to mature hypothalamus neurons.

In some embodiments, the ability to give rise to mature hypothalamus neurons is measured by detecting expression of one or more neuropeptide markers. Suitable neuropeptide markers include, but are not limited to, Otp, Rax, neuropeptide Y (NPY), cocaine amphetamine regulated transcript (CART), α-melanocyte stimulating hormone (α-MSH), neuropeptide Y receptor Y2 (NPYR), ghrelin receptor (GhrR), and melanin concentrating hormone (MCH).

In some embodiments, the one or more foreign molecules with which the exosomes are engineered comprise one or more of a growth factor, a nucleic acid, a cytokine, a vaccine, an inactivated viral protein, a drug, a chemotherapeutic and a biologically active molecule.

In some embodiments, the one or more nucleic acids with which the exosomes are engineered comprise DNA, messenger RNA, micro RNA, or small interfering RNA.

In some embodiments, the one or more growth factors with which the exosomes are engineered comprise human growth hormone (hGH), platelet-derived growth factor-BB (PDGF-BB), or bone morphogenetic protein (BMP).

In some embodiments, the one or more cytokines with which the exosomes are engineered comprise an interleukin, an interferon, or a synthetic cytokine.

In some embodiments, the one or more biologically active compounds with which the exosomes are engineered or loaded comprise one or more of a corticosteroid receptor agonist, an inhibitor of TNF-alpha, an inhibitor of NF-kB, an inhibitor of PI3K, a small molecule inhibitor of cytokine signaling, a small molecule antagonist of cIAP1/2 and XIAP (X-linked inhibitor of apoptosis protein), a mitochondria-derived activator of caspase (SMAC) mimetic; AT406, an inhibitor of IKK Complex, an inhibitor of Nuclear Factor Kappa-B Kinase Subunit Gamma (IKKγ) inhibitors, an IKKε and Tank Binding Kinase 1 (TBK1) inhibitor, a cytokine inhibitor, a hormone receptor agonist, a hormone receptor antagonist, NF-κB Inducing Kinase (NIK) inhibitor, a nuclear receptor agonist, a nuclear receptor antagonist, an inhibitor of ubiquitin-proteasome system, a proteasome inhibitor, NAE (NEDD8 activating enzyme) inhibitor, a deubiquitination (DUB) inhibitor, a molecule inhibiting nuclear translocation, a DNA binding and transcriptional activator of NF-κB, polyphenols-like curcumin, capsaicin, apigenin, oleandrin, quercetin, resveratrol, cinnamaldehyde, and epigallocatechin-3-gallate.

In some embodiments, the disease or disorder is one or more of an infection, a viral disease, a degenerative disease, an autoimmune disease, a genetic disorder, a dermatological disorder, a topical inflammation, a metabolic syndrome, a cancer, or a damaged tissue in need of repair.

In some embodiments, the disorder is tissue damage from a degenerative or ischemic disease. For example, the disease is dementia, neurodegenerative disease, high blood pressure, heart disease, cognitive decline from aging, Alzheimer's disease, Parkinson's disease, stroke, epilepsy, migraine, multiple sclerosis, neuropathy, spinal cord injury, traumatic brain injury, hearing loss, eye blindness, alcoholism, alcohol withdrawal, alcoholic neuropathy, neuropathic pain, multiple sclerosis, amyotrophic lateral sclerosis, Huntington's disease, ischemic brain injury, musculoskeletal trauma, over-use injury, age-related degeneration, cartilage tissue degeneration, arthritis, osteoarthritis, degenerative joint disease, rheumatoid arthritis, psoriatic arthritis, infectious septic arthritis, osteoporosis, a fracture, bone fracture, vertebral compression fracture, bone remodeling, dermal disorder, wound, prolonged inflammation, free radical damage, apoptosis, necrosis, coronary artery disease, myocardial infarction, blinding eye disease, age-related macular degeneration, metabolic syndrome, diabetes, polycystic ovary syndrome, fatty liver disease, cholesterol gallstones, asthma, sleep disturbance, or cancer.

In some embodiments, the pharmaceutical composition is in form of pill, capsule, tablet, gel, bead, lozenge, dragee, granule, aerogel, crumble, snap, liquid, powder, nebulizer, infusion, cream, lotion, depot, food product or wound healing composition.

In some embodiments, the pharmaceutical composition is administered to the subject by intracranial, oral, parenteral, topical, mucosal, sub-mucosal, pulmonary, nasal, or transdermal administration.

In some embodiments, the pharmaceutical composition may be administered to the subject in a daily therapeutically effective dosage of at least 0.01 mg, 0.1 mg, 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 100 mg, or 200 mg of engineered or loaded exosomes/kg of the subject.

In some embodiments, the pharmaceutical composition is administered to the subject at least 1, 2, 3, or 4 weeks prior to the disease or disorder onset, within 24 after the disease or disorder onset, or at least 1, 2, 3, or 4 weeks after the disease or disorder onset, in single or multiple doses.

In some embodiments, the pharmaceutical composition may be co-administered with one or more bioactive agents. Suitable bioactive agents include, but are not limited to, one or more of an antiviral agent, an anti-inflammatory agent, a hormone, a chemotherapeutic, a neural agent, or a pro-drug.

In some embodiments, the subject is a mammal. In some embodiments, the subject is a human subject.

The methods provided herein present several attractive features and desirable properties that make them suitable for use. For example, the disclosed method allows detection and purification of human hypothalamus stem cells at a precise stage during pluripotent or embryonic stem cell differentiation, when expression of hypothalamus markers and neuronal stem cell markers reaches its pick. The disclosed method is therefore precise, reliable and quick, as it reproducibly detects and isolates hypothalamus stem cells, and it requires less purification and validation steps. Furthermore, the disclosed method is easily scalable for large-scale production of human hypothalamus stem cells and exosomes. In addition, because disorders associated with the neuroendocrine system and the control of behavioral and physiological processes can be targeted by the disclosed human hypothalamus stem cell and exosome compositions, optimal treatment plans can be effectively devised and developed.

The foregoing and other features of the disclosure will become more apparent from the following detailed description of several embodiments, which proceeds with reference to the accompanying figures.

### BRIEF DESCRIPTION OF THE FIGURES

FIG. 1 shows a hypothalamus stem and progenitor cell isolation process scheme as provided herein. Pluripotent cells are cultured in media until they develop neuronal and hypothalamic differentiation factors. These factors allow the identification of cells with stem cell properties and the production of mature arcuate hypothalamus neurons.

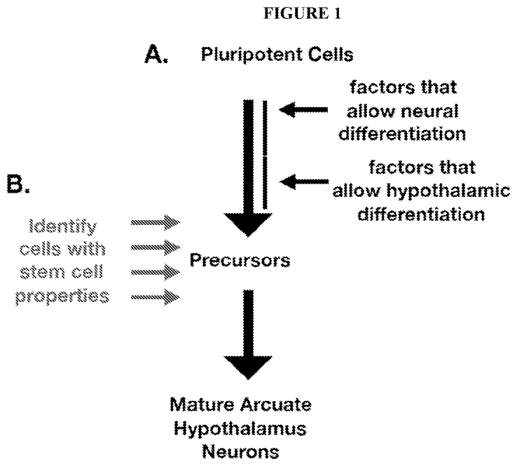

FIG. 2 shows the morphology of the induced pluripotent stem cell line WTC11, which can be differentiated into hypothalamus stem and progenitor cells by the process provided herein.

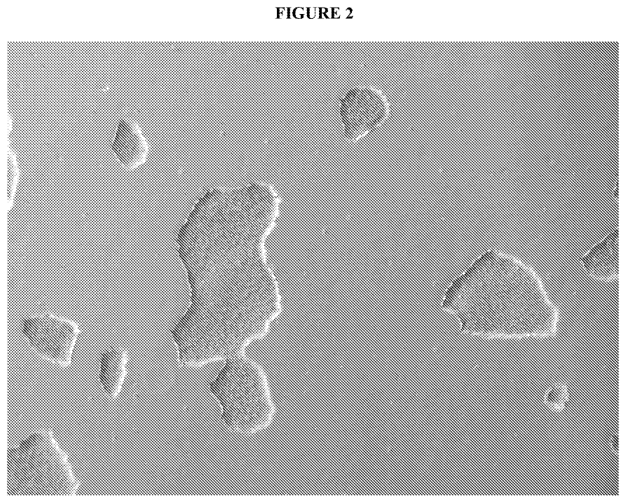

FIG. 3 shows the morphology of the induced pluripotent stem cell line WTC11, which can be differentiated into hypothalamus stem and progenitor cells by the process provided herein.

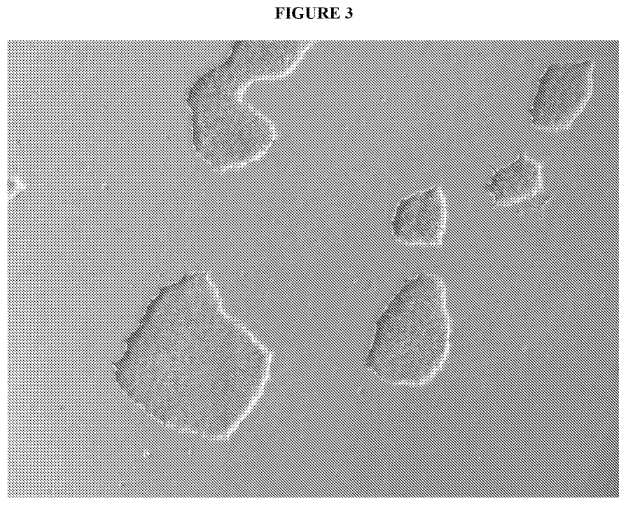

FIG. 4 shows the morphology of the induced pluripotent stem cell line WTC11, which can be differentiated into hypothalamus stem and progenitor cells by the process provided herein.

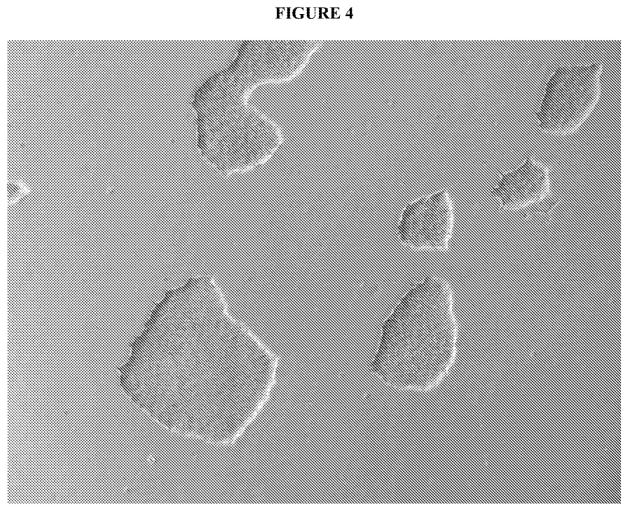

FIG. 5 shows the morphology of the induced pluripotent stem cell line WTC11, which can be differentiated into hypothalamus stem and progenitor cells by the process provided herein.

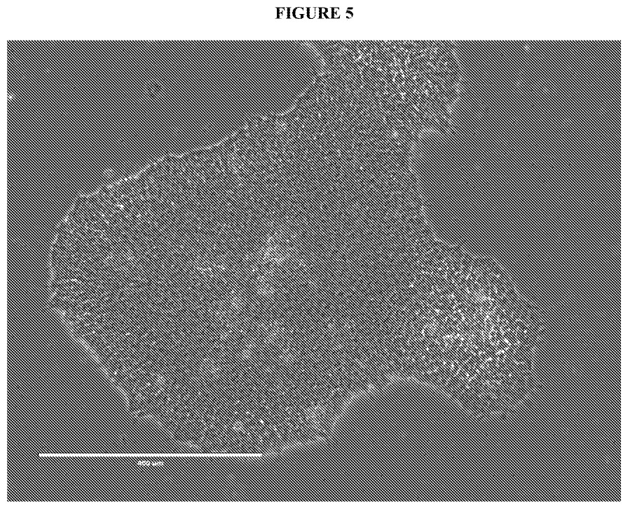

FIG. 6 shows a hypothalamus stem and progenitor cell isolation process scheme as provided herein. Embryonic stem cells or pluripotent cells are induced to differentiate in culture media under conditions that stimulate cell differentiation toward a hypothalamus cell lineage, such as Sonic HedgeHog activation (SHH) signaling pathway and SMAD and NOTCH signaling pathway inhibition, until the cells begin to express neuronal and hypothalamic differentiation markers. Cells that express markers of both neuronal stem/progenitor cells and of the hypothalamus cell lineage are identified, collected and used to produce mature arcuate hypothalamus neurons, and extracellular vesicles and exosomes thereof.

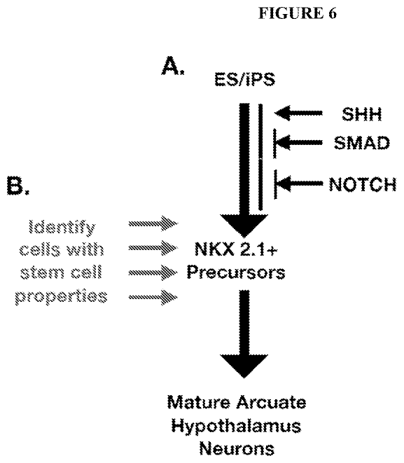

FIG. 7: (A) Human hypothalamus stem cells created by the disclosed method contain markers of both neural stem cells and cells of the hypothalamus lineage, as shown by immunofluorescence. (B) In contrast, undifferentiated parental pluripotent stem cells do not contain markers of neural stem cells or cells of the hypothalamus lineage, as shown by the lack of immunofluorescence.

FIG. 8: (A) Human hypothalamus stem cells created by the disclosed method contain markers of both neural stem cells and cells of the hypothalamus lineage, as shown by immunofluorescence. (B) In contrast, undifferentiated parental pluripotent stem cells do not contain markers of neural stem cells or cells of the hypothalamus lineage, as shown by the lack of immunofluorescence.

FIG. 9: Human hypothalamus stem cell exosomes are labeled using fluorescent dyes and detected. (A) Fluorescently labeled human hypothalamus stem cell exosomes are quantified using fluorescent nanoparticle tracking analysis (fNTA). (B) Liposomes containing 0.1 μM fluorescent polystyrene beads are used as control.

FIG. 10 shows RNA contained in human hypothalamus stem cell exosomes is stained with SYTO RNASelect stain.

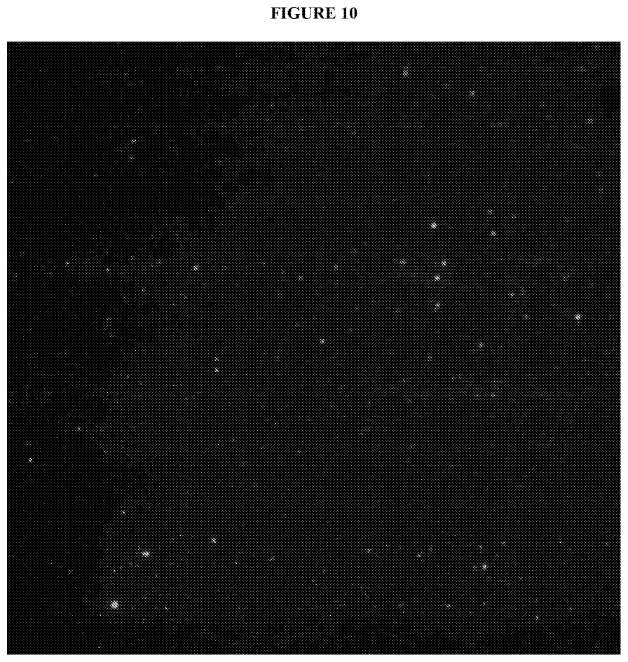

FIG. 11 shows the membrane of human hypothalamus stem cell exosomes stained with BODIPY TR ceramide.

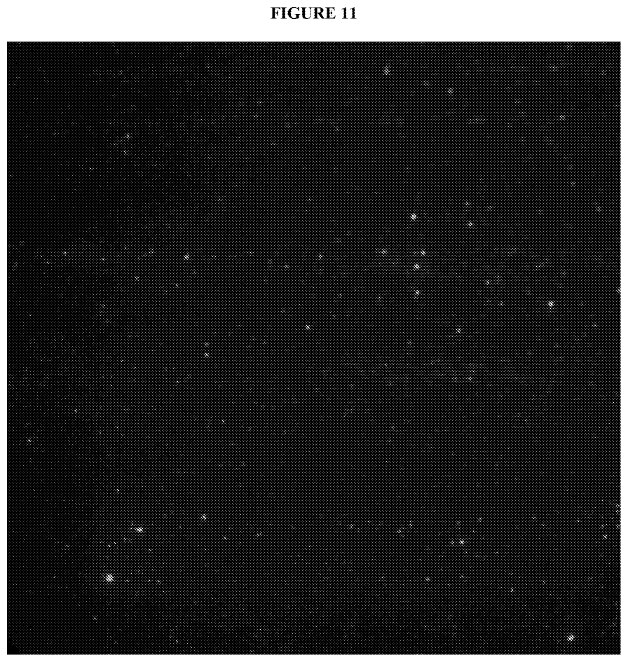

FIG. 12 shows the staining of both RNA and exosomal membrane in exosomal particles isolated from human hypothalamus stem cells. The double staining of both RNA and exosomal membrane provides evidence that the vesicles isolated from human hypothalamus stem cells are in fact exosomes, and not artifacts.

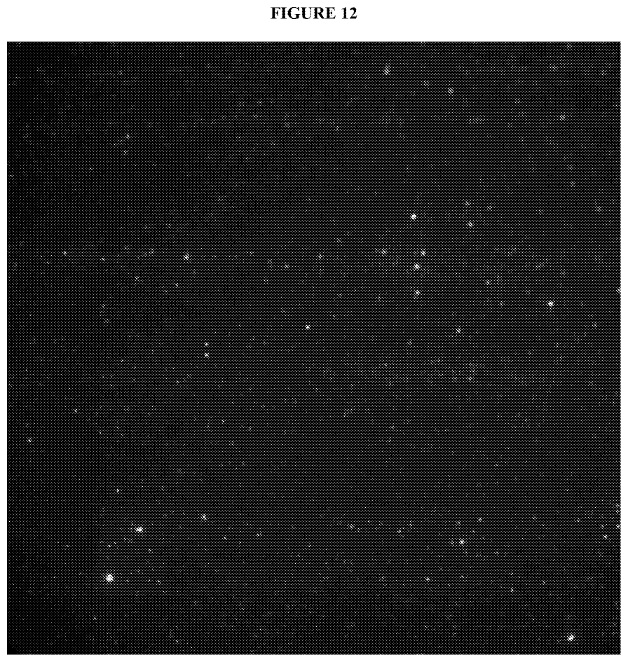

FIG. 13 shows transmission electron microscopy (TEM) images of human hypothalamus stem cells produced by the disclosed method.

FIG. 14 shows transmission electron microscopy (TEM) images of human hypothalamus stem cells produced by the disclosed method.

FIG. 15 shows transmission electron microscopy (TEM) images of human hypothalamus stem cells produced by the disclosed method.

FIG. 16 shows transmission electron microscopy (TEM) images of human hypothalamus stem cells produced by the disclosed method.

FIG. 17: capillary western blotting images of human hypothalamus stem cells produced by the disclosed method show that exosomes produced from human hypothalamus stem cells contain proteins associated with the exosomes.

FIG. 18 shows that human hypothalamus stem cell exosomes produced by the disclosed method reduce the expression of inflammatory genes in human cells.

FIG. 19 shows that human hypothalamus stem cells loaded with the thyroid hormone T3 activate the thyroid hormone receptor, as measured by qPCR of the thyroid hormone receptor target gene ectonucleotide pyrophosphatase/phosphodiesterase 2 (ENPP2).

FIG. 20 shows fluorescent staining of exogenous siRNA loaded into human hypothalamus stem cells, indicating that human hypothalamus stem cells were successfully engineered to express exogenous siRNA molecules.

FIG. 21 shows that human hypothalamus stem cells engineered to express IL-6 siRNA silence the expression of IL-6 in human cells.

FIG. 22 shows that human hypothalamus stem cell exosomes loaded with various NFKB inhibitors are able to cross an in vitro model of a blood brain barrier and suppress activation of NFKB. Negative control: vehicle control: HSC Exosome: human hypothalamus stem cell exosomes; Loaded exosome A: human hypothalamus stem cell exosomes loaded with NFKB inhibitor II-CAS 749886-87-1; Loaded exosome B: human hypothalamus stem cell exosomes loaded with NFKB inhibitor III-CAS 380623-76-7; Loaded exosome C: human hypothalamus stem cell exosomes loaded with NFKB inhibitor IV-CAS 139141-12-1.

### DETAILED DESCRIPTION OF THE INVENTION

The following explanations of terms and methods are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. For example, reference to “comprising a therapeutic agent” includes one or a plurality of such therapeutic agents. The term “or” refers to a single element of stated alternative elements or a combination of two or more elements, unless the context clearly indicates otherwise. For example, the phrase “A or B” refers to A, B, or a combination of both A and B.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

In some examples, the numbers expressing quantities of ingredients, properties such as molecular weight, reaction conditions, and so forth, used to describe and claim certain embodiments are to be understood as being modified in some instances by the term “about” or “approximately.” Notwithstanding that the numerical ranges and parameters setting forth the broad scope of some examples are approximations, the numerical values set forth in the specific examples are reported as precisely as practicable. The recitation of ranges of values herein is merely intended to serve as a shorthand method of referring individually to each separate value falling within the range.

To facilitate review of the various embodiments of this disclosure, the following explanations of specific terms are provided:

Administer: To provide or give a subject a composition by an effective route. Application is local. Exemplary routes of application include, but are not limited to, oral and topical routes.

Adult Stem Cell or Somatic Stem Cell: An undifferentiated cell found in a differentiated tissue and capable of renewing itself and under specific conditions differentiate into a specialized cell type of the tissue from which it originated. Adult stem cells are multipotent. Non-limiting examples of adult stem cells include hematopoietic stem cells, bone marrow-derived stem cells, neural stem cells (NSC), and multipotent stem cells derived from epithelial and adipose tissues and umbilical cord blood (UCB).

Antibiotic: A chemical substance capable of treating bacterial infections by inhibiting the growth of, or by destroying existing colonies of bacteria and other microorganisms.

Anti-inflammatory agent: An active agent that reduces inflammation and swelling.

Anti-Oxidant: An active agent that inhibits oxidation or reactions promoted by oxygen or peroxides.

Cell Differentiation: A process by which a less specialized cell, such as a stem cell, develops or matures or differentiates into a more specialized cell or a differentiated cell.

Conditioned Medium: A growth medium that contains soluble factors from cells cultured in the medium.

Contacting: Placement in direct physical association; includes both in solid and liquid form.

Control: A reference standard. In some examples, a control is a known value or range of values, such as one indicative of the presence or the absence of a disease. In some examples, a control is a value or range of values, indicating a response in the absence of a therapeutic agent.

Effective amount: The amount of an active agent (alone or with one or more other active agents) sufficient to induce a desired response, such as to prevent, treat, reduce and/or ameliorate a condition. Effective amounts of an active agent, alone or with one or more other active agents, can be determined in many different ways, such as assaying for a reduction in of one or more signs or symptoms associated with the condition in the subject or measuring the level of one or more molecules associated with the condition to be treated.

Embryonic Stem Cell: A cell derived from an embryo at the blastocyst stage or before substantial differentiation of the cell into the three germ layers that displays morphological characteristics of undifferentiated cells, and that is capable of self-renewing. Exemplary morphological characteristics of undifferentiated cells include, but are not limited to, high nuclear/cytoplasmic ratios and the presence of prominent nucleoli under a microscope. Under appropriate conditions, embryonic stem cells can differentiate into cells or tissues that are derivatives of each of the three germ layers: endoderm, mesoderm, and ectoderm. Assays for the identification of an embryonic stem cell include, but are not limited to, the ability to form teratoma in a suitable host and to be stained for markers of an undifferentiated cell, such as Oct-4.

Exosome or Extracellular Vesicle (EV): An extracellular vesicle that contains constituents of the cell that secretes it. Exosomes have a size from about 10 nm to about 10 m and consist of fluids, macromolecules, solutes, and metabolites derived from the cells that secrete them. They are contained within a lipid bilayer or micelle. Exosomes or extracellular vesicles may also include lipid vesicle engineered to contain bioactive molecules, such as a neurons, as well as ectosomes. Exosomes are released on the exocytosis of multivesicular bodies (MVBs). Ectosomes are vesicles assembled at and released from the plasma membrane. The size of the extracellular vesicles may be in a range from about 20 nm to about 10 m, or from about 20 nm to about 1 m, or from about 20 nm to about 500 nm, or from about 30 nm to about 100 nm, or from about 30 nm to about 160 nm, or from about 80 nm to about 160 nm. In some embodiments, the EVs are exosomes that are from about 20 nm to about 150 nm in size.

Induced Pluripotent Stem Cells (iPS or iPSCs): Pluripotent stem cells artificially generated from a non-pluripotent cell, typically an adult somatic cell, or from a terminally differentiated cell, such as a fibroblast, a hematopoietic cell, a myocyte, a neuron, an epidermal cell, or the like. Human induced pluripotent stem cells are produced from somatic cells, such as dermal fibroblasts, taken from a human individual. Pluripotent stem cells cells typically display the characteristic morphology of human embryonic stem cells (hESCs), express the pluripotency-associated markers SSEA-4 and TRA1-60, the transcription factors Oct-4 and Nanog, and differentiate in vitro into cell types derived from each of the three embryonic germ layers. Pluripotent stem cells may be an established cell line produced from somatic cells taken from a subject, or derived from any human somatic cell. Suitable somatic cells include, but are not limited to, keratinocytes, dermal fibroblasts and leukocytes derived from peripheral blood.

Inhibiting a condition: Reducing, slowing, or even stopping the development of a condition, for example, in a subject who is at risk of developing or has a particular condition.

Localized application: The application of an active agent in a particular location in the body.

Mucosal Administration: Administration through the mouth, nose, vagina, eyes and ears of a subject.

Oral administration: Delivery of an active agent through the mouth.

pH Adjuster or Modifier: A molecule or buffer used to achieve desired pH control in a formulation. Exemplary pH modifiers include acids (e.g., acetic acid, adipic acid, carbonic acid, citric acid, fumaric acid, phosphoric acid, sorbic acid, succinic acid, tartaric acid, basic pH modifiers (e.g., magnesium oxide, tribasic potassium phosphate), and pharmaceutically acceptable salts thereof.

Pharmaceutically acceptable carriers: The pharmaceutically acceptable carriers useful in this disclosure are conventional. The nature of the carrier can depend on the particular mode of administration being employed. For instance, oral applications usually include pharmaceutically and physiologically acceptable fluids such as water, physiological saline, balanced salt solutions, aqueous dextrose, glycerol or the like as a vehicle. In addition to biologically-neutral carriers, oral compositions may also contain minor amounts of non-toxic auxiliary substances, such as wetting or emulsifying agents, preservatives, and pH buffering agents and the like.

Stem Cell: A cell having the potential to self-renew and indefinitely divide, and, under appropriate conditions, differentiate into a dedicated progenitor cell or a specified cell or tissue. Stem cells can be pluripotent or multipotent. Stem cells include, but are not limited to embryonic stem cells, embryonic germ cells, cloned stem cells from somatic nuclei, adult stem cells, and umbilical cord blood cells. The term “stem cells” encompasses mammalian stem cells, including human stem cells, and non-human mammalian stem cells, including, but not limited to, those derived from primates, ungulates, ruminants and rodents. When cultured under suitable conditions and/or contacted with, or exposed to, particular compounds and/or conditions, stem cells may differentiate into any one of the specialized cell types which form embryonic and/or adult tissues.

Subject: A living multi-cellular vertebrate organism, a category that includes human and non-human mammals, as well as birds (such as chickens and turkeys), fish, and reptiles. Exemplary subjects include mammals, such as human and non-human primates, rats, mice, dogs, cats, rabbits, cows, pigs, goats, horses, and the like.

Surface or Body Surface: A surface located on the human body or within a body orifice. Thus, a “body surface” includes, by way of example, skin, teeth, skin or mucosal tissue, including the interior surface of body cavities that have a mucosal lining.

Tanycytes: Cells in the hypothalamus that have stem cell properties.

Topical administration: Delivery of an active agent to a body surface, such as, the skin or mucosa, as in, for example, topical drug administration in the prevention or treatment of various skin disorders.

Under conditions sufficient to: A phrase that is used to describe any environment that permits the desired activity.

Method for Producing Human Hypothalamus Stem Cells and Exosomes from Induced Pluripotent or Embryonic Stem Cells

The hypothalamus is a small, but essential region of the brain. Hypothalamic neurons respond to physiological stimuli by releasing neurotransmitters and neuromodulators into the brain, and regulate several physiological and behavioral functions, including immune and hormonal function, reproduction, stress, circadian rhythms, sleep pattern, mood status and social behavior.

Hypothalamus dysfunction has been linked to metabolic and inflammatory diseases, such as obesity and neuro-inflammation, and to sleep and mood disorders. In vitro generation of hypothalamic stem cells and neurons is vital for the study and understanding of these diseases and disorders, and for the development of appropriate therapies. The unavailability of human hypothalamic stem cells and inconsistently reproducible protocols are major obstacles to progressive research in this field.

Provided herein is an approach that overcomes these challenges, by providing a reliable, cost-effective and easily scalable method that allows isolation and purification of human hypothalamus stem cells and exosomes at a precise intermediate stage during induced pluripotent or embryonic stem cell in vitro differentiation, just as the developing hypothalamus stem cells reach maximal expression of hypothalamus markers and neuronal stem cell markers maximal expression. The disclosed method is highly efficient, as hypothalamic stem cells can be grown, isolated and purified in a short time, between day 7 and day 15 of cell culture, and preferably at day 8 or day 9 of cell culture. The hypothalamic stem cells produced in vitro by the method provided herein are morphologically similar to hypothalamic stem cells produced in vivo, and can be used for the production of exosomes and the development of drug screens and therapies for hypothalamus-related diseases and disorders.

Thus, provided herein is a method for identifying, isolating and purifying human hypothalamus stem cells at an intermediate transitory stage during in vitro induced pluripotent stem cell differentiation, which corresponds to human hypothalamus stem cell formation. The disclosed method comprises: (i) culturing induced pluripotent stem cell in culture media and enabling reproducible differentiation of induced pluripotent stem cells into cells of ventral diencephalon hypothalamic cell lineage; (ii) identifying hypothalamic cells between day 0 and day 15 of cell culture that display hypothalamus markers and neuronal stem cell markers; (iii) isolating hypothalamus stem cells at hypothalamus marker and neuronal stem cell marker maximal expression; and (iv) purifying isolated human hypothalamus stem cells.

To stimulate differentiation toward hypothalamic lineage and decrease cell variability, the pluripotent or embryonic stem cells are cultured in presence of small molecules and peptide modulators that block the BMP and the TGF-β pathways, and the canonical Wnt pathways.

Human hypothalamus stem cell formation is identified by detecting the expression of neuronal stem cell markers, such as Sox2⁺ and Bmi-1⁺; hypothalamus markers, such as NK2 homeobox 1 (Nkx2.1) and homeobox protein orthopedia; Rax^{hi} and Sox1^{lo}; and proteins that are mainly expressed in nerve cells, such as neuroectodermal stem cell marker ((nestin)⁺, Musashi RNA binding protein 1 (Musashi1)⁺ and C-X-C motif chemochine receptor 4 (Cxcr4)*.

The disclosed method may further comprise analyzing the purified human hypothalamus stem cells for immunophenotype, neurosphere formation and ability to give rise to mature hypothalamus neurons.

In some embodiments, the ability to give rise to mature hypothalamus neurons is measured by detecting expression of one or more neuropeptide markers. Suitable neuropeptide markers include, but are not limited to, Otp, Rax, neuropeptide Y (NPY), cocaine amphetamine regulated transcript (CART), α-melanocyte stimulating hormone (α-MSH), neuropeptide Y receptor Y2 (NPYR), ghrelin receptor (GhrR), and melanin concentrating hormone (MCH).

Additionally provided herein is the production of exosomes from hypothalamic stem cells. Thus, in some embodiments, the disclosed method may further comprise isolating and purifying exosomes from the stem cell culture media, and analyzing the purified exosomes by one or more suitable techniques, such as, for example, western blot analysis, flow cytometry, nanoparticle-tracking analysis, density-gradient ultracentrifugation, pull-down assay, and real-time PCR.

The cells producing the exosomes may be cultured on a layer of feeder cells, on a cellular support or matrix, as adherent monolayers, as embryoid bodies or in suspension. Cellular supports may comprise at least one substrate protein. Suitable substrate proteins include, but are not limited to, an extracellular matrix protein, such as laminin, tenascin, thrombospondin, and mixtures thereof, collagen, fibronectin, vibronectin, polylysine, polyornithine and mixtures thereof; and gels and other materials such as methylcellulose.

The disclosed exosomes are harvested at various time intervals, such as at about 2, 4, 6, 8 days, or 3, 6, 9, 12 days, or longer intervals, depending upon the rate of production of exosomes. Exemplary yields of exosomes may range from at least about 1 ng exosomes/1 million cells, to at least about 10 ng exosomes/1 million cells, to at least about 50 ng exosomes/1 million cells, to at least about 100 ng exosomes/1 million cells, to at least about 500 ng exosomes/1 million cells, to at least about 750 ng exosomes/1 million cells, to at least about 800 ng exosomes/1 million cells, to at least about 900 ng exosomes/1 million cells, to at least about 1.0 μg exosomes/1 million cells, to at least about 1.5 μg exosomes/1 million cells, to at least about 2.0 μg exosomes/1 million cells, to at least about 2.5 μg exosomes/1 million cells, to at least about 3.0 μg exosomes/1 million cells, to at least about 5.0 μg exosomes/1 million cells, to at least about 10.0 μg exosomes/1 million cells, during a time period of about 24 hours to seven days of culture of proliferative and non-proliferative neural cells.

In some embodiments, the exosomes are harvested and collected by ultracentrifugation or differential centrifugation or any combination thereof, and the pelleted exosomes are collected, and, optionally, washed with a suitable medium. For example, a preparation of exosomes can be prepared from a cell culture or tissue supernatant by centrifugation, filtration or combinations of these methods. In some embodiments, the exosomes can be prepared by differential centrifugation at low speed (<2,0000 g) to pellet larger particles, followed by high-speed (>100,000 g) centrifugation to pellet exosomes. The exosomes are then size filtered with appropriate filters (for example, 0.22 μm filter), gradient ultracentrifugation (for example, with sucrose gradient) or a combination of these methods. The exosomes may be purified by differential centrifugation, micro and ultrafiltration, polymeric precipitation, microfluidic separation, immunocapture and size-exclusion chromatography. Other methods for isolation may be developed, such as electrical field radiofrequency and acoustics.

Exosome purification and exosome enrichment of preparations may be performed by any suitable method, such as filtration, dialysis, ultracentrifugation, and/or chromatography. In some embodiments, polyethylene glycol precipitation and/or chromatographic enrichment using the monolithic technology as stationary phases may be used. Monoliths are continuous stationary phases that are cast as a homogeneous column in a single piece and prepared in various dimensions with agglomeration-type or fibrous microstructures.

Fractions enriched with exosomes are tested for their in vitro potency effect, in particular for their ability to modulate gene expression, regulate hypothalamic function, deter aging, slow down neurodegeneration, musculoskeletal degeneration, or other biological functions. In addition, these fractions are analyzed for protein content and particle size.

Alternatively, the cells may be cultured in a culture system that is essentially free of feeder cells, but nonetheless supports proliferation of the cells to produce exosomes. The growth of cells in feeder-free culture can be supported using a conditioned or a chemically-defined medium.

Method for Producing Therapeutics and Compositions Thereof from Human Hypothalamus Stem Cell-Derived Exosomes for the Treatment of Diseases

Infection with an infectious agent or a virus often induces a cascade of short, intermediate and long-term effects that are detrimental to an organism. High fatality rates caused by viral infections, such as infections with SARS-CoV-2, the 1918 H1N1 influenza virus, the Epstein-Barr virus or the Ebola virus, are thought to be due partly to the onset of a cytokine storm in the advanced stages of the infection, when a large number of pro-inflammatory cytokines, such as IL-1, IL-6, and TNF-α, and anti-inflammatory cytokines, such as IL-10, are released, with consequent hypotension, hemorrhage, and, ultimately, multi-organ failure. Cytokine storm is also correlated with acute pancreatitis, severe burns, trauma, acute respiratory distress syndrome secondary to drug use and inhalation of toxins.

Autoimmune conditions potentially related to viral infections include, but are not limited to, multiple sclerosis, systemic lupus erythematosus, rheumatoid arthritis and Sjogren's syndrome. Cytomegalovirus and herpes simplex virus are associated with coronary artery conditions.

The ability of the body to regenerate tissue damaged by trauma or degeneration is critical to health. Current strategies for tissue replacement are lacking in that they do not foster the ability of the body to regenerate its own tissue. In addition, the ability of musculoskeletal tissue to regenerate itself declines with age, and can be further compromised by trauma induced by overuse or injury. Degenerative joint diseases, such as osteoarthritis, rheumatoid arthritis, psoriatic arthritis, and infectious septic arthritis, osteoporosis and metabolic changes associated with aging are caused by a combination of factors, including genetic factors, nutritional imbalance, lack of exercise, and excessive exercise or injury. Similarly, dermal disorders, wound healing, and intractable skin ulcers, are medical conditions that would benefit from improved tissue regeneration. Metabolic syndrome is associated with the risk of developing cardiovascular disease, diabetes, and other conditions such as polycystic ovary syndrome, fatty liver disease, cholesterol gallstones, asthma, sleep disturbances, and some forms of cancer. The ability to regenerate bone, joint and dermal tissue has the potential to improve the lives of millions of humans, however, current strategies to prevent the decline of dermal, musculoskeletal and joint tissue in degeneration or trauma are lacking.

Improving neuronal tissue regeneration would benefit multiple disorders of various etiology such as: spinal cord injury, stroke, traumatic brain injury, hearing loss, blinding eye diseases, alcoholism and alcohol withdrawal, alcoholic neuropathy, or neuropathic pain as well as neurodegenerative diseases such as multiple sclerosis, Alzheimer's disease, Alzheimer's disease related disorders, Amyotrophic Lateral Sclerosis, Parkinson's disease, and Huntington's disease. The development of efficient treatment for these diseases remains major public health issues due to their incidence as well as lack of curative treatments. Despite active research in this area, there is still a need for alternative or improved efficient therapies for neurological disorders.

Effective therapies targeting viral inflammations, viral-induced conditions, neurogenerative diseases and related inflammatory conditions are urgently needed, as currently available methods of treatment are insufficient.

Provided herein are methods and compositions for making therapeutics from neuronal stem and progenitor cell exosomes, and their use for the attenuation, reduction, management, control and/or treatment of diseases, infection and disorders brought about by infectious agents, such as viruses and bacteria, inflammation, cancer, trauma, shock, stroke, neurodegenerative diseases, drug overdoses, stimulant abuse, seizure, pregnancy complications, progressive neurological disorders, genetic disorders, and drug withdrawal symptoms.

Thus, provided herein are pharmaceutical compositions comprising purified human hypothalamus exosomes obtained as described above and engineered to express one or more foreign molecules for in vitro and in vivo targeted delivery of active compounds. Suitable foreign molecules include, but are not limited to, growth factors, nucleic acids, cytokines, vaccines, inactivated viral proteins, drugs, chemotherapeutics and biologically active molecules.

Suitable nucleic acids include, but are not limited to, DNA, messenger RNA, micro RNA, or small interfering RNA (siRNA), such as siRNA molecules targeting one or more genes, such as toll-like receptor 4 (TLR-4), NFkappaB, and tumor necrosis factor alpha; interleukins, such as interleukin-1 and interleukin-6; proteins, such as fatty acid binding protein Fh 15; and interferons, such as IFN-alpha and IFN-gamma.

Suitable growth factors include, but are not limited to, growth factors involved in organism development, angiogenesis and wound healing, such as human growth hormone (hGH), platelet-derived growth factor-BB (PDGF-BB), and bone morphogenetic protein (BMP).

Suitable cytokines include, but are not limited to, interleukins, interferons, and synthetic cytokines.

The engineered exosomes are formulated into pharmaceutical compositions and administered to a subject in need thereof in order to prevent, treat, manage or control a hypothalamus-related disease or disorder, an infection, such as a viral, bacterial or cancer-related infection, an autoimmune disease, a genetic disease, a coronary disease, a respiratory disease, a kidney disease, trauma, shock, stroke, a drug overdose, stimulant abuse, seizure, pregnancy complications, a progressive neurological disorder, a genetic disorder, drug withdrawal symptoms or a pathology in the subject.

Additionally, the disclosed exosomes may be loaded with small chemical compounds for targeted delivery of biologically active compounds into cells, tissues and organs. Exemplary small molecules that can be delivered by the disclosed engineered exosomes include, but are not limited to, corticosteroid receptor agonists, such as dexamethasone and cortisol, prednisolone and vamorolone, ulinastatin, chloroquine, eritoran, thalidomide, and didox; inhibitors of TNF-alpha, such as TNF-α inhibitor CAS 1049741-03-8; inhibitors of NF-kB, such as NF-kB activation inhibitor IV-CAS 139141-12-1, KINK-1 hydrochloride, Bay 11-7082, pacritinib, R-835, PF-06650833, and AU-4948; inhibitors of PI3K; small molecule inhibitors of cytokine signaling; small molecule antagonist of cIAP1/2 and XIAP (X-linked inhibitor of apoptosis protein), such as ASTX660; mitochondria-derived activator of caspase (SMAC) mimetic; AT406 (Debio-1143); inhibitors of IKK Complex, such as SAR-113945, MLN-0415, AS-602868, CHS-828, VGX-1027, teglarinad Chloride (EB-1627; GMX1777, and sulfasalazine; inhibitor of Nuclear Factor Kappa-B Kinase Subunit Gamma (IKKγ) inhibitors; IKKε and Tank Binding Kinase 1 (TBK1) inhibitors; cytokine inhibitors; hormone receptor agonists; hormone receptor antagonists; NF-κB Inducing Kinase (NIK) inhibitors; nuclear receptor agonists; nuclear receptor antagonists; inhibitors of ubiquitin-proteasome system; proteasome inhibitors; NAE (NEDD8 activating enzyme) inhibitors; deubiquitination (DUB) inhibitors; molecules inhibiting nuclear translocation; DNA binding and transcriptional activation of NF-κB, polyphenols-like curcumin, capsaicin, apigenin, oleandrin, quercetin, resveratrol, cinnamaldehyde, and epigallocatechin-3-gallate.

Suitable methods for loading purified exosomes include, but are not limited to, transfection, electroporation, expression of fusion proteins that bind the therapeutic during exosome biogenesis, and the like. Exosomes can also be loaded by directly engineering the hypothalamus stem cells to express a recombinant construct as described above.

Suitable diseases or disorders correlated with hypothalamic malfunction that can be prevented, treated, managed or controlled by the administration of engineered stem cells and exosomes produced according to the disclosed method include, but are not limited to, infectious diseases, including pandemic influenza, pandemic respiratory diseases, COVID-19, COVID-19-related diseases, long-term complications of COVID-19, SARS-Co-V2 and variants thereof, and Ebola; functions mediated by vasopressin or antidiuretic hormone, such as high blood pressure, cardiac function, trauma, bleeding disorders, kidney diseases, aberrant plasma osmolarity, diabetes, syndrome of inappropriate antidiuretic hormone (SIDAH), anxiety, PTSD, hemorrhages, septic shock, gastrointestinal bleeding, bedwetting, hyponatremia, hypothyroidism, lethargy, fatigue, loss of appetite, irritability, muscle weakness, spasms or cramps, seizures, and decreased consciousness or coma; functions mediated by the hormone oxytocin, such as postpartum disorders, sexual disorders, autism, anxiety, psychological trauma, post-traumatic stress disorders, and panic disorders; functions mediated by growth hormone-releasing hormone (GHRH) or growth hormone-inhibiting hormone (GHIH), such as acromegaly and growth hormone deficiencies, such as Turner syndrome, Prader-Willi syndrome, brain tumors, acquired growth hormone deficiency (AGHD), delayed puberty or altered sexual development, reduced bone strength, depression, lack of concentration, memory deficiencies; functions mediated by dopamine dysregulation, such as Parkinson's disease, restless leg syndrome, sleep disorders, pain, emotional disorders, muscle cramps, spasms, tremors, aggressive behavior, compulsive behaviors, ADHD, binge eating, gambling addiction, psychosis, schizophrenia, bipolar disorder, Huntington's disease, and Tourette's syndrome; functions mediated by thyrotropin-releasing hormone, thyroid stimulating hormone, thyroxine and triiodothyronine, such as hypothyroidism, thyroid disorders, mood swings, fatigue, lack of sex drive, diarrhea, breast cancer, spinal cord injury, amyotrophic lateral sclerosis, spinocerebellar degenerative disease, regulation of insulin production, function of β-cells of the pancreas, thyroid-induced cognitive, neurological and fertility disorders, and immune function; functions mediated by adrenocorticotropic hormone, corticotropin-releasing hormone (CRH) and cortisol, such as aberrations of the hypothalamic-pituitary-adrenal axis, inflammatory diseases, auto-immune diseases, acute respiratory distress syndrome, Cushing's syndrome, and adrenal insufficiency; conditions affecting the pituitary gland, sleep disorders, dysfunctions of the circadian rhythm, hypopituitarism, congenital adrenal hyperplasia, Nelson's syndrome, adrenoleukodystrophy, West syndrome (“infantile spasms”), postorgasmic illness syndrome (POIS), DAVID syndrome, osteoporosis, skin disorders, muscle weakness, amenorrhoea, panic disorder, eating disorders, insomnia, and narcolepsy; functions mediated by gonadotropin-releasing hormone (GnRH), luteinizing hormone, follicle-stimulating hormone or prolactin, prolactin-releasing hormone (PRH) or prolactin-inhibiting hormone (PIH or dopamine), such as menstrual disorders, menopause symptoms and alterations, infertility, in vitro fertilization, endometriosis, prostate cancer, hypogonadotropic hypogonadism, transgender hormone therapy, hot flashes, suppression of spontaneous ovulation as part of controlled ovarian hyperstimulation, hyperandrogenism, hair loss, female androgenetic alopecia, and veterinary medicine; conditions directly or indirectly related to the hypothalamus, such as regulation of circadian rhythms, sleep disorders, mood disorders, physiological functions, fibrillations, Adams-Stokes disease, sinus arrhythmia, Wolff-Parkinson-White syndrome, ventricular tachycardia, slurred speech, decreased motor function, abnormal breathing patterns, fever, hyperthermia, hypothermia, sweating, constipation, irritable bowl disorders, Crohn's disease, ulcerative colitis, pancreatitis, Celiac disease, atherosclerosis, aneurysm, Raynaud's disease, thromboembolism, erythromelalgia, pulmonary embolism, coronary artery disease, carotid artery disease, varicose veins, vasculitis, cancer, substance abuse, and hormone secretion; neurological disorders, such as delirium, including ICU delirium, ventilator-induced delirium, and Covid-19-induced delirium, mental health disorders, including attention deficit hyperactivity disorder (ADHD), adjustment disorder, agoraphobia, Alzheimer's disease, amnesia, amphetamine dependence, anorexia, depression, bipolar disorder, bulimia nervosa, caffeine-induced anxiety disorder, caffeine-induced sleep disorder, cannabis dependence, claustrophobia, cocaine dependence, dementia, depersonalization disorder, dissociative identity disorder, Down syndrome, drug withdrawal, dyslexia, hysteria, impulse control disorder, kleptomania, Korsakoff's syndrome, language disorder, learning disorders, Munchausen syndrome, narcissistic personality disorder, neurodevelopmental disorder, nicotine dependence, nightmare disorder, obsessive-compulsive disorder, oneirophrenia, onychophagia, orthorexia nervosa, paranoia, pedophilic disorder, pyromania, reactive attachment disorder, reading disorder, REM sleep behavior disorder, sadistic personality disorder, selective mutism, separation anxiety disorder, Stendhal syndrome, stereotypic movement disorder, Stockholm syndrome, tardive dyskinesia, vaginismus and voyeuristic disorder; and recreation and preventative health use, including mood enhancement, inducement of pleasure, feelings of wellbeing and vitality, improvement of virility and sexual function in males and females, motivation increase, ability to concentrate on a task, cognitive ability, intelligence and creativity, slowing down aging; cytokine storm, septic shock, multiorgan dysfunction syndrome, septicemia, systemic inflammatory response syndrome, disseminated intravascular coagulation, hypovolemia, stroke, reperfusion injury after stroke, burn trauma, full-thickness burns, post-burn reaction, thermal regulation disorders, severe hypothermia, high altitude illness, acute mountain sickness, high altitude cerebral edema, high altitude pulmonary edema, drug withdrawal symptoms, drug overdose symptoms, drug toxicity and side effects, including side effects such as seizures, hypothermia, hyperthermia, hypotension, hypertension, cardiac dysrhythmias, respiratory depression, ataxia, anxiety, blurred vision, headache, methamphetamine withdrawal symptoms, rhabdomyolysis, excited delirium, malignant hyperthermia, hyperthermia from use or abuse of stimulants such as ecstasy/MDMA, cocaine, methamphetamine, and amphetamines, visual disturbances, eclampsia, pre-eclampsia, epilepsy, seizure disorder, vascular dementia, Lewy body syndrome, fronto-temporal dementia, Huntington's disease, acidosis, alkalosis, anaerobic respiration, cancer, and pregnancy complications.

The disclosed engineered or loaded exosomes are formulated into pharmaceutical compositions for oral, parenteral, intracranial, pulmonary, topical, transdermal, mucosal or sub-mucosal administration.

The compositions provided herein may be in solid, liquid, gel, semi-solid, lyophilized or powder forms, such as, for example, solutions, suspensions, emulsions, sustained-release formulations, tablets, capsules, powders, suppositories, creams, ointments, lotions, aerosols, patches or the like, in unit dosage forms suitable for administration of precise dosages. The pharmaceutical compositions may comprise therapeutically effective amounts of the disclosed exosomes and one or more pharmaceutically acceptable excipients, such as carriers, adjuvants, additives, flavors and the like. The weight percentage ratio of the disclosed exosomes to the one or more excipients can be from about 20:1 to about 1:60, or from about 15:1 to about 1:45, or from about 10:1 to about 1:40, or from about 9:1, 8:1, 7:1, 6:1, 5:1, 4:1, 3:1, 2:1 or 1:1 to about 1:2, 1:3, 1:4, 1:5, 1:6, 1:7, 1:8, 1:9, 1:10, 1:15, 1:20, 1:25, 1:30, or 1:35. In some embodiments, the disclosed pharmaceutical compositions may comprise from about 1 μg to about 1 g or more of total exosomes, or from about 500 μg to about 500 mg of total exosomes, or from about 1 mg to about 500 mg of total exosomes, or from about 5 mg to about 500 mg of total exosomes, or from about 10 mg to about 500 mg of total exosomes, or from about 25 mg to about 500 mg of total exosomes, or from about 50 mg to about 350 mg of total exosomes, or from about 75 mg to about 450 mg of total exosomes, or from about 50 mg to about 450 mg of total exosomes, or from about 75 mg to about 325 mg of total exosomes, or from about 100 mg to about 650 mg of total exosomes.

The disclosed pharmaceutical compositions may be formulated in immediate release form, sustained release form or controlled release form, and coated using compounds that accelerate or decrease the release of the active ingredient. Thus, the disclosed compositions may comprise enteric coatings, extended-release coatings, sustained-release coatings, delayed release coatings and immediate-release coatings. Methods used to coat compositions as well as the materials used to manufacture such coatings are well known in the pharmaceutical formulary art. Coating materials may include, but are not limited to, glyceryl monostearate, glyceryl distearate, polymeric substances and waxes.

The disclosed compositions may be combined with additional active ingredients as needed. For example, the pharmaceutical compositions may comprise one or more of a neurotrophic agent, leukemia inhibitory factor (LIF), brain-derived neurotrophic factor (BDNF), epidermal growth factor receptor (EGF), basic fibroblast growth factor (bFGF), FGF-6, glial-derived neurotrophic factor (GDNF), granulocyte colony-stimulating factor (GCSF), hepatocyte growth factor (HGF), IFN-γ, insulin-like growth factor binding protein (IGFBP-2), IGFBP-6, IL-1ra, IL-6, IL-8, monocyte chemotactic protein (MCP-1), mononuclear phagocyte colony-stimulating factor (M-CSF), neurotrophic factors (NT3), tissue inhibitor of metalloproteinases (TIMP-1), TIMP-2, tumor necrosis factor (TNF-β), vascular endothelial growth factor (VEGF), VEGF-D, urokinase plasminogen activator receptor (uPAR), bone morphogenetic protein 4 (BMP4), IL1-a, IL-3, leptin, stem cell factor (SCF), stromal cell-derived factor-1 (SDF-1), platelet derived growth factor-BB (PDGFBB), transforming growth factors beta (TGFβ-1) and TGFβ-3.

The disclosed exosomes may be formulated with a biocompatible scaffold, such as a hydrogel. Suitable hydrogels include temperature-dependent hydrogels that solidify or set at body temperature, crosslinked hydrogels, and the like, that comprise cross-linked polysaccharides, such as alginate, polyphosphazenes, and polyacrylates, or block copolymers, such as poly(oxyethylene)-poly(oxypropylene) block polymers. Suitable hydrogels also include undefined extracellular matrix-derived hydrogels that originated from tissues including but not limited to bladder intestine, blood and brain. Suitable biocompatible scaffolds include, but are not limited to, collagen, fibrin, silk, agarose, alginate, hyaluronan, chitosan, a biodegradable polyester such as polylactic-co-glycolic acid, polylacic acid, or polyglycolic acid, polyethylene glycol, polyvinylpyrrolidone, polyethersulfone, a peptide-based biomaterial, glycose amino glycan, fibronectin, laminin, and any combination thereof.

The disclosed pharmaceutical compositions may be administered to a subject in need thereof in a daily therapeutically effective dosage of about 0.01, 0.1, 1, 5, 10, 15, 20, 25, 30, 35, 40, 45, 50, 55, 60, 65, 70, 75, 80, 85, 90 or 100 mg, or greater than 100, 110, 120, 130, 140, 150, 160, 170, 180, 190 or 200 mg of engineered or loaded exosomes/kg of the subject receiving the compositions.

In some embodiments, the disclosed exosome compositions are administered at least 1, 2, 3, or 4 weeks before infection, within 24 after infection with an infectious agent, or at least 1, 2, 3, or 4 weeks after infection, in single or multiple doses.

The disclosed exosomes may be loaded with small molecules, antisense oligonucleotides, siRNAs, peptides, proteins or antibodies that target peptides or peptide translation products involved in immunomodulation. In some embodiments, the disclosed exosomes may be loaded with additional bioactive agents or are co-administered with additional bioactive agents for co-administration or combination therapy of two or more active compounds or compositions to treat a neural injury and/or a neurodegenerative disease. Co-administration may include simultaneous administration, or administration from about one minute to several minutes, to about eight hours, or from about 30 minutes to about 6 hours, or from about an hour to about 4 hours of two or more compounds or compositions.

Exemplary antiviral agents that may be administered intravenously or orally in combination with the disclosed exosomes include, but are not limited to, pyrimidine nucleoside analogs, such as ddl, ddC, AZT and FIAU (fluoro-iodo-arabinofuranosyl-uracil), and purine nucleoside analogs, such as acyclovir, ribavirin, ganciclovir, and vidarabine.

The disclosed exosomes may be co-administered with a glucocorticoid. Suitable glucocorticoids include, but are not limited to. medrysone, alclometasone, alclometasone dipropionate, amcinonide, beclometasone, beclomethasone dipropionate, betamethasone, betamethasone benzoate, betamethasone valerate, budesonide, ciclesonide, clobetasol, clobetasol butyrate, clobetasol propionate, clobetasone, clocortolone, cloprednol, Cortisol, cortisone, cortivazol, deflazacort, desonide, desoximetasone, desoxycortone, desoxymethasone, dexamethasone, diflorasone, diflorasone diacetate, diflucortolone, diflucortolone valerate, difluorocortolone, difluprednate, fluclorolone, fluclorolone acetonide, fludroxycortide, flumetasone, flumethasone, flumethasone pivalate, flunisolide, flunisolide hemihydrate, fluocinolone, fluocinolone acetonide, fluocinonide, fluocortin, fluocoritin butyl, fluocortolone, fluorocortisone, fluorometholone, fluperolone, fluprednidene, fluprednidene acetate, fluprednisolone, fluticasone, fluticasone propionate, formocortal, halcinonide, halometasone, hydrocortisone, hydrocortisone acetate, hydrocortisone aceponate, hydrocortisone buteprate, hydrocortisone butyrate, loteprednol, meprednisone, 6a-methylprednisolone, methylprednisolone, methylprednisolone acetate, methylprednisolone aceponate, mometasone, mometasone furoate, mometasone furoate monohydrate, paramethasone, prednicarbate, prednisolone, prednisone, prednylidene, rimexolone, tixocortol, triamcinolone, triamcinolone acetonide and ulobetasol.

Solid dosage forms suitable for oral administration may include, but are not limited to, capsules, tablets, pills, powders, beads, lozenges, dragees, granules, aerogels, crumbles, snaps, or the like. Such solid dosage forms may include at least one pharmaceutically acceptable excipient or carrier, such as sodium citrate or dicalcium phosphate; fillers or extenders, such as starches, lactose, sucrose, glucose, mannitol, and silicic acid; binders, such as, for example, carboxymethylcellulose, alginates, gelatin, polyvinylpyrrolidone, sucrose, and acacia; humectants, such as glycerol; disintegrating agents such as agar-agar, calcium carbonate, potato or tapioca starch, alginic acid, silicates and sodium carbonate; solution retarding agents such as paraffin; absorption accelerators, such as quaternary ammonium compounds; wetting agents such as, for example, acetyl alcohol and glycerol monostearate; absorbents such as kaolin and bentonite clay; lubricants, such as talc, calcium stearate, magnesium stearate, solid polyethylene glycols, sodium lauryl sulfate, and mixtures thereof, and buffering agents.

Solid oral dosage forms may also be formulated as dietary compositions, and may comprise any ingestible preparation that contains the disclosed therapeutics mixed with a food product. The food product can be dried, cooked, boiled, lyophilized or baked, and may be in the form of breads, cookies, teas, juices, soups, cereals, salads, sandwiches, sprouts, vegetables, candies, pills, tablets, or the like.

Liquid dosage forms for oral administration may include, but are not limited to, pharmaceutically acceptable emulsions, solutions, suspensions, syrups and elixirs, and may contain inert diluents commonly used in the art. For instance, liquid formulations may contain water, polyethylene glycol ethers, or any other pharmaceutically acceptable solvents; solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, and dimethyl formamide; oils, such as cottonseed, groundnut, corn, germ, olive, castor, and sesame oils; glycerol, tetrahydrofurfuryl alcohol, polyethylene glycols and fatty acid esters of sorbitan; adjuvants, such as wetting agents; emulsifying and suspending agents, such as ethoxylated isostearyl alcohols, polyoxyethylene sorbitol, sorbitan esters, microcrystalline cellulose, aluminum metahydroxide, bentonite, agar-agar, tragacanth, and mixtures thereof; sweetening, flavoring, perfuming agents, and any mixture thereof.

Liquid oral dosage forms may also be formulated as dietary compositions, and may comprise any ingestible preparation that contains the disclosed therapeutics mixed with a drink product. Drink products may include, but are not limited to, teas, juices, syrups, soups, sodas, brewed drinks, fermented drinks, distilled drinks, or the like.

Parenteral administration may include subcutaneous injections, intravenous, intramuscular, intrasternal injection or infusion techniques. Suspensions for parenteral administration may be encapsulated with a variety of polymers, sugars, and chelating agents, to yield stable preparations or granules. Polymers for encapsulation may include crosslinked polymers, non-crosslinked polymers, or polymers dispersed within the crystalline structure of sugar starches or protein molecules.

Compositions for parenteral injection may comprise pharmaceutically acceptable sterile aqueous or nonaqueous solutions, dispersions, suspensions or emulsions as well as sterile powders for reconstitution into sterile injectable solutions or dispersions prior to use. Examples of suitable aqueous and nonaqueous carriers, diluents, solvents or vehicles include, but are not limited to, water, ethanol, polyols, such as glycerol, propylene glycol, polyethylene glycol, and the like, carboxymethylcellulose and suitable mixtures thereof, vegetable oils, such as olive oil, and injectable organic esters, such as ethyl oleate. Proper fluidity can be maintained, for example, by the use of coating materials such as lecithin, by the maintenance of the required particle size in the case of dispersions, and by the use of surfactants. Compositions for parenteral administration may also contain adjuvants such as, but not limited to, preservatives, wetting agents, emulsifying agents, and dispersing agents, isotonic agents, such as sugars, sodium chloride, and the like, and agents that delay absorption, such as aluminum monostearate and gelatin.

The disclosed pharmaceutical compositions may comprise about 50 ng, 100 ng, 200 ng, 300 ng, 400 ng, 500 ng, 600 ng, 700 ng, 800 ng, 900 ng, 1.0 μg, 1.5 μg, 2.0 μg, 2.5 μg, 3.0 μg, 5.0 μg, 10.0 μg, 15.0 μg, 20.0 μg, 100 μg, or more exosomes/ml intravenous/intrathecal/intracerebrospinal fluid medium for use in treating spinal cord injury, stroke, traumatic brain injury and/or neurodegenerative diseases.

Intravenous formulations may comprise about 0.1 μg, about 0.2 μg, about 0.3 μg, about 0.4 μg, about 0.5 μg, about 0.6 μg, about 0.7 μg, about 0.8 μg, about 0.9 μg, about 1.0 μg, about 1.5 μg, about 2 μg, about 2.5 μg, about 3 μg, about 4.0 μg, about 5.0 μg, about 10 μg, about 15 μg, or about 20 μg or more of exosomes/ml intravenous medium.

Topical compositions may be in form of powder, solution, emulsion, suspension, cream, salve, gel, or gum gel, and can be applied to the face, eyes, lips, teeth, hair, forehead, nails, hands, feet, shoulders, arms, back, or legs of a subject.

Transdermal compositions may be in form of patch, wound dressings, bandages, plasters, stents, implants, aerogels, crumbles, snaps, or hydrogel for transdermal application, and formulated for immediate release, extended release or sustained release. Various additives, known to those skilled in the art, may be included in transdermal formulations. Examples of additives include, but are not limited to, solubilizers, skin permeation enhancers, preservatives, such as anti-oxidants, moisturizers, gelling agents, buffering agents, surfactants, emulsifiers, emollients, thickening agents, stabilizers, humectants, dispersing agents and pharmaceutical carriers. Examples of moisturizers include, but are not limited to, jojoba oil and evening primrose oil. Suitable skin permeation enhancers include, but are not limited to, lower alkanols, such as methanol ethanol and 2-propanol; alkyl methyl sulfoxides, such as dimethylsulfoxide (DMSO), decylmethylsulfoxide (C10 MSO) and tetradecylmethyl sulfoxide; pyrrolidones, urea; N,N-diethyl-m-toluamide; C2-C6 alkanediols; dimethyl formamide (DMF), N,N-dimethylacetamide (DMA) and tetrahydrofurfuryl alcohol. Examples of solubilizers include, but are not limited to, hydrophilic ethers, such as diethylene glycol monoethyl ether and diethylene glycol monoethyl ether oleate; polyoxy 35 castor oil, polyoxy 40 hydrogenated castor oil, polyethylene glycol (PEG), and polyethylene glycol derivatives, such as PEG-8 caprylic/capric glycerides; alkyl methyl sulfoxides, such as DMSO; pyrrolidones, DMA, and mixtures thereof.

Prevention and/or treatment of infections can be achieved by the inclusion of antibiotics, as well as various antibacterial and antifungal agents, for example, paraben, chlorobutanol, phenol sorbic acid, and the like, in the disclosed compositions.

The disclosed compositions may also be administered by a variety of other routes, including intracranial, mucosal, subcutaneous and intramuscular administration, and may comprise a variety of carriers or excipients known in the formulary art, such as non-toxic solid, semisolid or liquid filler, diluent, encapsulating material and formulation auxiliaries that are pharmaceutically acceptable.

In additional embodiments, provided herein is a method for identifying a drug that modulates hypothalamus function in a subject in need thereof. The disclosed method comprises (a) transfecting human hypothalamus stem cells with a reporter gene operably linked to a transcriptional regulatory element isolated from the subject and regulating expression of the reporter gene; (b) contacting transfected human hypothalamus stem cells with a candidate drug; (c) determining drug effect on transcriptional regulatory element expression by detecting and measuring a signal emanating from the reporter gene and comparing the signal to a signal emanated by the reporter gene prior to contact with the drug, thereby identifying a drug that modulates hypothalamus function in the subject.

In some embodiments, the effect of the drug may be further determined by measuring one or more properties, such as cell viability, proliferation, apoptosis, stem-ness, and differentiation.

Suitable candidate drug include compounds that decrease neuroinflammation, chemotherapeutics, and compounds that alter circadian rhythm, regulate thyroid function or affect obesity, inflammation, infertility or aging.

The subject can be a mammal, such as an animal or a human subject.

The disclosed methods are reliable and quick, and easily scalable for large-scale production of human hypothalamus stem cells and exosomes. In addition, the disclosed human hypothalamus stem cell and exosome compositions are suited to specifically target disorders associated with the neuroendocrine system and the control of behavioral and physiological processes in different subjects, and allow the development of optimal treatment plans for personalized therapy.

The following examples illustrate the disclosed methods for producing and using human hypothalamus stem cells and exosomes thereof according to the present invention. These examples are illustrative only and are not intended to limit the scope of the invention as defined by the claims presented herein.

### EXAMPLES

### Example 1: Identification and Isolation of Human Hypothalamus Stem Cells During Induced Pluripotent Stem Cell Differentiation

It is not known at what stage of pluripotent cell differentiation human hypothalamus stem cells are formed. The aim of this project was to identify and isolate human hypothalamus stem cells at the very intermediate stage of their formation during induced pluripotent stem cell differentiation. The underlying hypothesis was that during the in vitro iPSCs differentiation process, the pluripotent stem cells give rise to progeny that passes through a number of intermediate cell types, only one of which is the hypothalamus stem cell stage. Each progeny contains intermediate stem cells with increasingly restricted differentiation potential as they mature into hypothalamus cells. The goal was, therefore, to identify for the first time the exact stage at which hypothalamus stem cell differentiation occurs.

### Pluripotent Stem Cell Culture

Cultured cells are incubated at 37° C. in 5% CO₂/20% O₂. Mouse embryonic stem cells are maintained under feeder-free conditions on gelatin-coated plates in medium containing KO-DMEM, 10% knockout serum replacement, 2 mM glutamax, MEM-NEAA, 1 mM sodium pyruvate, 55 μM β-mercaptoethanol, about 2000 units/ml LIF, 0.5 μM PD0325901, and 3 μM CHIR99021. Mouse embryonic stem cells (mESCs) are passaged at 20% confluence and are used at passage 40 or lower. Human embryonic stem cells (hESCs) and human induced pluripotent stem cells (hiPSCs) are maintained on plates coated with hESC-qualified Matrigel in chemically defined mTeSR-1 medium, and are passaged by manual picking or by enzymatic digestion with TrypLE Express in the presence of 10 μM Y27632. Human embryonic stem cell lines used for most experiments are HUES48, HUES49 or HUES53, and hiPSC lines are hiPS17a and hiPS18b. HESCs are passaged at 80% confluence and are used at passage 40 or lower. Media for both mouse and human stem cells are changed daily.

### Self-Patterning to Hypothalamic Neurons

Human PSCs are enzymatically dissociated to a single cell suspension with TrypLE Express, pelleted and resuspended in mTeSR-1 medium with 10 μM Y27632. Cells are plated into Lipidure-coated round-bottomed 96-well plates at a concentration of 5000 hPSCs per well. The following day (D0), cell aggregates are formed at the bottom of the wells and mTeSR is removed by four washes of GMEM, followed by two washes with growth factor-free chemically defined medium (gfCDM) containing 7 g/ml insulin and 2 μM Akt inhibitor VIII. Cells are maintained in 150 μl of this medium per well for 30 days and fed with half-volume media changes (75 μl) every 2 days. Owing to evaporation, the wells nearest to the edge of the plate are excluded from analysis. On D30, cell aggregates are washed three times with KSR medium and gradually switched to maturation medium with half-volume medium changes every 2 days. Maturation medium consisted of Neurobasal-A, 2 mM glutamax-I, 1×N2 supplement, 1×B27 supplement, 0.075% sodium bicarbonate, 200 nM ascorbic acid, 1 μM dibutyryl cyclic AMP (dbcAMP), and 10 ng/ml each of GDNF, BDNF and CNTF. Cell aggregates are maintained as free-floating aggregates until analysis on D90, or are transferred to monolayers of cortical glia as either whole cell aggregates or as dissociated cells. Cell aggregates are dissociated by enzymatic digestion in papain and DNAseI for 20 minutes at 37° C., followed by mechanical trituration, passage through a 40 m cell strainer, pelleting and plating at a density of 10-100,000/cm² in maturation medium containing 10 μM Y27632.

Directed Differentiation of hPSCs to Hypothalamic Neurons

HPSCs are dissociated to single cells and plated on matrigel in mTeSR1 containing 10 μM Y27632 at a density of 70,000 cells/cm². The following day (D0), mTeSR1 is replaced with KSR medium containing 10 μM SB431542, 100 nM LDN193189, and 2 μM XAV939. KSR medium is gradually replaced with N² medium every other day from D4-D8. To ventralize the resulting putative prosencephalic progenitors, 1 μM purmorphamine and 1 μM SAG are added from D2 to D8. On D30, cultures enzymatically dissociated are re-plated onto a monolayer of cortical astrocytes at a density of 0.5-5×10⁵ cells/cm² in maturation medium. Cells are maintained under these conditions by half-volume media changes every other day.

### Pluripotent Stem Cell Differentiation

Pluripotent stem cells grown under conditions that favor the differentiation of mature hypothalamus cells are treated with proteolytic and collagenolytic enzymes to dissociate cell clusters into single cells, and plated in 6 well-coated plates at a density of approximately 1×10⁶ cells/well in E8 medium with 10 μM of Rho-associated protein kinase (ROCK) inhibitor Y27632 overnight. Cell differentiation is then initiated by dual SMAD inhibition using 1 μM LDN193189 and 10 μM SB431542 for 48 hours in DMEM/F12 medium supplemented with 1×SLDM supplement (containing 200 ml DMEM/F12, 125 mg/ml bovine serum albumin, 27.15 mg/ml sodium bicarbonate, 3.2 mg/ml L-ascorbic acid, 805 μg/ml putrescine, 750 μg/ml D(+)-galactose, 250 μg/ml holo-transferrin, 125 μg/ml catalase, 125 μg/ml L-carnitine, 50 μg/ml reduced glutathione, 0.7 μg/ml sodium selenite, 50 μg/ml ethanolamine, 0.1 μg/ml T3 (triiodo-L-thyronine), 1 μg/ml corticosterone, 50 μg/ml linoleic acid, 50 μg/ml linolenic acid, 2.35 μg/ml lipoic acid (thioctic acid), 0.32 μg/ml progesterone, 5 μg/ml retinol acetate, 50 μg/ml a-tocopherol (vitamin E), and 50 μg/ml a-tocopherol acetate), 1% PSA (Anti-anti), 1× glutamax, 2.5 μg/ml superoxide dismutase, 4 μg/ml insulin, matrigel and vitronectin.

The cell cultures are then subjected to dual SMAD inhibition with LDN193189, a BMP type I receptor inhibitor of activin receptor like kinases (ALKs) ALK2 and ALK3, and SB431542, a transforming growth factor-beta 1 (TGF β1) of ALK-4, -5 and -7, followed by sonic hedgehog activation with 1 μM smoothened agonist SAG and 1 μM purmorphamine, and by Wnt signaling inhibition with 10 μM IWR-endo from day 2 to day 9 to direct cell differentiation towards ventral diencephalon. The media is regularly replaced every two days. On days 2-8 of culture, small molecule pathway modulators of sonic hedgehog (Shh) (smoothened agonist (SAG), purmorphamine (PMN), and inhibition of Wnt//β-catenin signaling (CHIR99021 and IWR-1-endo)) are added to the cultures to stimulate differentiation of the cells towards ventral diencephalon hypothalamic cell identity. Cell cultures are harvested on day 0 and then every day up to day 15 or beyond to determine the presence of hypothalamic stem cells in the culture. Cultures are analyzed for immunophenotype, neurosphere formation and ability to give rise to mature hypothalamus neurons. Hypothalamic cells are identified at day 9 by detection of the NK2 homeobox 1 (Nkx2.1) and the homeobox protein orthopedia (Otp; hypothalamic neuron progenitor that specifies neuropeptidergic neurons).

Cells are defined as hypothalamus stem cells if they display one or more cell surface antigens whose presence is characteristic of hypothalamus cells: specifically Nkx2.1^{hi}, Rax^{hi}, Sox1^{lo}, as well as mouse neuronal stem cell antigens Sox2+ and Bmi-1+. Hypothalamus stem cells may be further defined as being nestin⁺, Musashi1⁺ and Cxcr4⁺. A gradient is observed, where expression of hypothalamus markers gradually increases beginning on day 0 and over time, whereas expression of neuronal stem cell markers reaches a pick and thereafter declines. Hypothalamus stem cells are identified and isolated when they reach maximal expression of hypothalamus markers and neuronal stem cell markers, while retaining their ability to self renew by forming neurospheres and differentiate into mature hypothalamus cells.

### Example 2: Differentiation of Human Hypothalamus Stem Cells into Mature Hypothalamus Neurons

To determine whether human hypothalamus stem cells are capable of developing into mature hypothalamus neurons, day 9 to day 15 stem cell growth is inhibited by addition of 10 μM DAPT and caudalization with 0.01 μM retinoic acid. On day 14, the cells are treated with proteolytic and collagenolytic enzymes to dissociate cell clusters into single cells, and the cells are re plated onto laminin-coated plates and maintained in medium containing 10 ng/ml brain-derived neurotrophic factor BDNF until day 40.

Detection of hypothalamic markers Otp, Rax, neuropeptide Y (NPY, a secreted neuropeptide of the orexigenic NPY neurons), cocaine amphetamine regulated transcript (CART, a neuropeptide produced by anorexigenic CART neurons), α-melanocyte stimulating hormone (α-MSH, a bioactive product of POMC producing neurons), neuropeptide Y receptor Y2 (NPYR), ghrelin receptor (GhrR, ghrelin response receptors present in ARC neurons), and melanin concentrating hormone (MCH, orexigenic hypothalamic peptide found in hypothalamic MCH neurons) indicates that human hypothalamus stem cells developed into mature hypothalamus neurons.

### Example 3: Neurosphere Formation

To obtain primary cultures of hypothalamus stem cells, the hypothalamus and hippocampus are dissected from human cadavers or non-human animals, cut into small pieces of approximately 1 mm, and enzymatically digested for 30 min at 37° C. The cells are centrifuged, suspended in neurobasal medium containing 0.24% L-alanine, L-glutamine supplement, 2% B27 without vitamin A, 10 ng/ml EGF, 10 ng/ml bFGF, and 1% penicillin-streptomycin, and seeded in ultralow adhesion 6-well plates for one week. Neurospheres are then collected by centrifugation, enzymatically digested into single cells, passaged and maintained in neurosphere culture until use.

### Example 4: Differentiation of Human Embryonic Stem Cells and Induced Pluripotent Cells into Neurons

Human embryonic stem (ES) cells or human induced pluripotent (hPS) cells are cultured on a layer of feeder cells or in a feeder-free system with no differences in hypothalamic differentiation. hPS cells are cultured in 6-well plates in the presence of mouse embryonic fibroblasts (MEF). The cells are then seeded on matrigel-coated plates (feeder-free) to initiate neuron differentiation once each well reaches 95-100% confluence. The hPS cells have uniform shape and actively proliferate upon starting differentiation. SB 431542 and LDN 193189 are added from day 1 to day 8 to inhibit transforming growth factor beta (TGFβ) and bone morphogenetic protein (BMP) signaling and promote neuron differentiation from human ES/iPS cells. Sonic hedgehog (SHH) signaling molecule and Purmorphamine (PMA) are also added from day 1 to day 8 to induce ventral brain development and NKX2.1 expression. Further inhibition of Notch signaling is obtained by addition of DAPT from day 9 to day 12 to increase NKX2.1 and activity-regulated cytoskeleton-associated protein (Arc) expression for neuron precursor formation.

6-well plates are seeded on day 0, and from day 0 to day 15 a cell culture is harvested every day. Hypothalamic stem cells are identified between day 0 and day 15 of culture by marker detection, immunophenotype analysis, visualization of neurosphere formation, and by detecting the presence of mature hypothalamus neurons in the culture, as described above. Specific marker detection includes detection of Nkx2.1^{hi}, Rax^{hi}, Sox1^{lo}, and neuronal stem cell antigens Sox2+ and Bmi-1+. Hypothalamus stem cells are further defined as being nestin⁺, Musashi1⁺ and Cxcr4⁺. A gradient is observed, where expression of hypothalamus markers gradually increases beginning on day 0 and over time, whereas expression of neuronal stem cell markers reaches a pick and thereafter declines. Hypothalamus stem cells are identified and isolated when they reach maximal expression of hypothalamus markers and neuronal stem cell markers, while retaining their ability to self renew by forming neurospheres and differentiate into mature hypothalamus cells.

### Example 5: Exosome Formation

Hypothalamus stem and progenitor cells are obtained as described above. Exosomes secreted into the culture medium are then purified by differential centrifugation at 4° C. overnight to remove impurities and generate a medium with a lesser content of exosomes. The cells are cultured in the exosome-impoverished medium for two days, after which the medium is collected, centrifuged to remove cells, and exosomes are immediately isolated at 4° C. The medium is centrifuged and filtered to remove debris, and the exosomes in the filtered medium are isolated by differential centrifugation. An analogous volume of uncultured medium is subjected to the same purification procedures and used as control. Isolated exosomes are analyzed by different approaches.

In a first approach, immunoblot analysis of exosomal protein markers is performed. Exosomal and cellular proteins are loaded and separated by SDS-PAGE, followed by western blotting analysis with primary antibodies: mouse anti-TSG101, rabbit anti-CD81, rabbit anti-AGO2, rabbit anti-HSP90B1, mouse anti-GM130, and rabbit anti-Cycl. The exosomes are then detected by silver staining. In a second approach, flow cytometry is performed. Purified exosomes are incubated with latex beads for 15 minutes, adjusted to a final volume, and rotated at room temperature for 2 hours. Glycine is added and after incubation, beads are collected by centrifugation, resuspended and stained by FACS using FITC-conjugated anti-CD81 antibody. In a third approach, nanoparticle-tracking analysis of the purified exosomes is performed. In a fourth approach, the exosomes are analyzed by density-gradient ultracentrifugation analysis. The isolated exosomes are resuspended in 2.5M sucrose solution (20 mM HEPES, pH 7.4), plated on a continuous sucrose gradient from 2M to 0.25M, and ultracentrifuged overnight at 4° C. Different fractions are collected, each fraction recovered by ultracentrifugation and analyzed by immunoblot and real-time PCR. In a fifth approach, the exosomes are analyzed by pull-down assay: anti-CD81 and isotype-control antibodies are coated onto Protein G beads, the exosomes are re-suspended in PBS containing 3 mg/ml BSA, and incubated with antibody-coated beads overnight with rotation at 4° C. The beads are subsequently washed with the same buffer, and the isolated exosomes are analyzed by real-time PCR. In a sixth approach, exosomal total protein and RNA are measured.

### Example 6: Collection of Conditioned Media and Purification of Exosomes

Conditioned media (CM) from cells cultured as described above is harvested every 48 hours and filtered through a 0.22 μm membrane to remove cell debris and larger vesicles. CM is collected and stored in batches at −20° C. until use. The CM batches are then thawed, combined and subject to PEG precipitation by incubation with PEG 6000 for 8-12 hours at 4° C., followed by differential centrifugation for 30 minutes at 1,500 g to obtain exosome pellets. The pellets are resolved in 1 ml 0.9% NaCl, washed in a total volume of 45 ml 0.9% NaCl for 12 hours at 4° C., and separated by ultracentrifugation for two hours at 100,000 g. The pellets are again resolved in 1 ml 0.9% NaCl, diluted in with 0.9% NaCl to reach the required concentrations, and stored as 1 ml aliquots at −80° C. until use.

### Example 7: Quality Control Assessment of Exosomes

The quality of the exosomes obtained as described above is assessed by determining the concentration of protein in each aliquot. For each aliquot, 10 μg of exosome-containing fraction is analyzed by Western blots using antibodies that recognize exosome-specific marker proteins: Tsg101, CD63, CD81 and/or CD82. The concentration and size of the exosomes is additionally assessed by nanoparticle tracking analysis, and qPCR is used to determine the content and, optionally, the bioactivity of the exosomes when administered to cells.

### Example 8: Isolation of Exosomes Produced from Bioreactors

Hypothalamus cells obtain from humans, veterinary sources, or derived from stem cells are grown in bioreactors.

### Cell Culture Method for Exosome Production

Exosomes are obtained by ultrafiltration of complete medium through a 500 kDa commercial hollow fiber ultrafiltration module. A peristaltic pump is used to slowly circulate culture medium through the filter module, and the filtrate is collected to be used as exosomes. The entire procedure is carried out using sterile materials within a biosafety cabinet. Ultrafiltered supernatant is filtered a second time through a 0.22 mm filter device to ensure sterility.

To obtain conditioned medium from conventional cultures, 3 million cells are seeded in 175 cm² tissue culture flasks in DMEM supplemented with 10% fetal calf serum and 100 U/mL penicillin/streptomycin. After overnight incubation, the cell monolayer is gently washed with PBS, and 15 ml of fresh exosome medium is added to each flask. 48 hours later, conditioned medium is harvested and pooled for immediate exosome purification.

### Fibercell Hollow-Fiber Bioreactor Culture

Hypothalamus cells are expanded in conventional culture flasks and used to seed a medium-sized, hollow-fiber culture cartridge, with a 20 kDa molecular weight cut-off. Cells are adapted over two weeks to bioreactor culture conditions by gradually increasing the proportion of protein-free medium (DMEM 10% Fibercell Systems CDMHD protein-free supplement 100 U/ml penicillin/streptomycin). Bioreactor conditioned medium (20 ml) is collected for each harvest three times per week. Harvests are cleared of cells by 300 g centrifugation, and supernatants stored at 80° C. for further purification.

### Example 9: Purification of Exosomes by Differential Centrifugation

Conditioned media (CM) from cells cultured as described above is harvested every 48 hours and filtered through a 0.22 μm membrane to remove cell debris and larger vesicles. Differential ultracentrifugation is used to separate the exosomes by their vesicle size and sedimentation properties. Sequential centrifugation steps with increasing force of centrifugation deplete the conditioned medium from large particles and/or vesicles with high sedimentation rates. A final ultracentrifugation (UC) step sediments small vesicles or exosomes, and leaves the smaller proteins in the supernatant.

The cell culture medium is replaced with exosome-depleted medium, which is obtained by centrifuging stem cell medium at 100,000 g for at least 17 hours, and incubated for 48 hours. The exosomes are then purified from the conditioned medium by differential UC. Cell debris is pelleted at 300 g for 10 minutes. Larger vesicles are pelleted at 10,000 g for 30 minutes, and the supernatant is filtered through a 0.2 μm membrane. The exosomes are then pelleted at 100,000 g for 90 minutes using 70 ml polycarbonate bottles and Type 45 Ti rotor. Exosome pellets are washed once in 1 ml sterile PBS and centrifuged for 90 minutes at 100,000 g in a tabletop ultracentrifuge using a TLA-110 rotor.

### Example 10: Purification of Exosomes by Tangential Flow Filtration

Conditioned media (CM) from cells cultured as described above is harvested every 48 hours and filtered through a 0.22 μm membrane to remove cell debris and larger vesicles. Exosomes are isolated and purified from three-dimensional cell cultures by growing the cells on the surface of spherical support matrix beads with continuous stirring. A pump circulates the conditioned culture medium through membranes or filters to remove small particles and maintain larger particles in optimal concentration. In particular, the conditioned cell culture supernatant is first passed through a 200-nm pore size membrane to remove large vesicles and particles.

The filtered conditioned medium is subjected to tangential flow filtration using a hollow fiber filter with a 500-kDa molecular weight cutoff (MWCO). A feed flow rate of 120 ml/min, trans-membrane pressure <3.5 psi, and a cross-flow rate >10:1 is maintained throughout the filtration operation. The conditioned medium is concentrated 9-fold and then replaced with 6× volume of PBS, by continuously feeding the system with PBS to replace the loss of permeate. The isolated exosomes are filtered through a 0.2 μm membrane and stored in 0.1M sucrose in polyethylene terephthalate glycol (PETG) bottles at −80° C.

### Example 11: Exosome Transfection

Exosomes produced as described above are engineered to express foreign nucleic acids, including siRNAs. 4.5×10¹⁰ exosomes are co-incubated with 1 nmol of siRNA at 37° C. for 1 hour in 500 μl of PBS to obtain a loading mixture. The exosome-siRNA mixture is centrifuged at 100,000 g for 90 minutes, and the supernatant containing unloaded siRNA is removed. The pellets are suspended in 500 μl of PBS to measure fluorescence, or in 300 μl of neural medium to treat primary neurons. To quantify loading of Cy3-labeled siRNA, a 200 μL aliquot is taken from the suspended exosome pellet or from the supernatant. Fluorescence is measured at 550 nm excitation, 570 nm emission. The percent loaded siRNA is calculated as pellet/(pellet+supernatant). The siRNA copy number per exosome is estimated as: (percent of loaded siRNA)×(amount of siRNA initially mixed with exosomes [mol])×(Avogadro number)/(number of exosomes initially mixed in).

For exosome transfection with microRNA, 4.5×10¹⁰ exosomes are co-incubated with 1 nmol of miRNA at 37° C. for 1 hour in 500 μl of PBS to obtain a loading mixture. The exosome-miRNA mixture is centrifuged at 100,000 g for 90 minutes, and the supernatant containing unloaded miRNAs is removed. The pellets are suspended in 500 μl of PBS to measure fluorescence, or in 300 μl of neural medium to treat primary neurons. To quantify loading of Cy3-labeled miRNA, a 200-μl aliquot is taken from the suspended exosome pellet or from the supernatant. Fluorescence is measured at 550 nm excitation, 570 nm emission. The percent loaded miRNA is calculated as pellet/(pellet+supernatant). The miRNA copy number per exosome is estimated as: (percent of loaded miRNA)×(amount of miRNA initially mixed with exosomes [mol])×(Avogadro number)/(number of exosomes initially mixed in).

### Example 12: Isolation of Human Hypothalamus Stem Cells and Exosomes from Humans

Hypothalamus cells are isolated from the hypothalamus region of the brain and from the median eminence of the third ventricle in human adult subjects and in young infants and children less than two years old, following brain resection surgery. Hypothalamus cells are also isolated from the same regions in cadavers.

The cells are placed in basal media supplemented with glutamine, insulin, nitrogen, B27 supplement, and growth factors, including EGF, FGF-2, IGF-1, BNDF, NT-3, DKK1, GNDF, laminin, and BMPR1a-Fc, to stimulate neural stem cell growth.

Cells that express both hypothalamic and stem cell markers, including Nestin, Sox-2 and Musashi, are selected, and those expressing a neural stem cell immunophenotype are separated using antibodies to the antigens ABCG2, CD133, CXCR4, FGF R4, Frizzled-9 Glut1, SSEA-1, Notch-1 and Notch-2.

The isolated cells are then immortalized, and human hypothalamus stem cell exosomes are collected as described above.

### Example 13: Identification of Drugs that Decrease Neuroinflammation for the Treatment of Diseases of the Hypothalamus

To identify drugs that decrease neuro-inflammation, the immortalized hypothalamus cells obtained as described above are transfected with a reporter gene operably linked to NFκB p50/p65, a transcriptional regulatory element, which is isolated from a target organism and regulating the expression of the reporter gene.

The transfected immortalized hypothalamus cells are contacted with a candidate drug, and the signal emanating from the reporter gene is detected and compared to the signal emanated by the reporter gene before exposure of the cells to the drug, to obtain the drug response profile.

### Example 14: Identification of Drugs that Alter the Circadian Rhythm for the Treatment of Sleep Disorders and Other Diseases of the Hypothalamus

To identify drugs that alter the circadian rhythm, the immortalized hypothalamus cells obtained as described above are transfected with a reporter gene operably linked to the promoter regions of the period (per) and timeless (tim) genes, or to the NR2f6 gene, and contacted with a candidate drug, to determine the effect of the drug on sleep disorders and the circadian rhythm in a subject.

### Example 15: Identification of Drugs for the Treatment of Thyroid Disorders

To identify drugs for the treatment of thyroid disorders, such as obesity and impaired metabolism, the immortalized hypothalamus cells obtained as described above are transfected with a reporter gene operably linked to the promoter regions of the thyroid hormone receptor gene, and contacted with a candidate drug, to determine the effect of the drug on thyroid disorders in a subject.

### Example 16: Identification of Drugs that Correct Human Hypothalamus Function for the Treatment of Diseases of the Hypothalamus

To identify drugs that correct human hypothalamus function, the immortalized hypothalamus cells obtained as described above are transfected with a reporter gene operably linked to one or more of the HMX2 (Nkx5-2), SIM1 (bHLHe14), PITX2 (ARP1), SOX14 (SOX28), HMX3 (Nkx5-1), SIX6 (OPTX2), DMBX1 (OTX3), EBF3 (COE3), NKX2-1(BCH), CITED1 (MSG1), ISL1 (Isl-1), and LHX5 genes.

The transfected immortalized hypothalamus cells are contacted with a candidate drug, and the signal emanating from the reporter gene is detected and compared to the signal emanated by the reporter gene before exposure of the cells to the drug, to obtain a personalized drug response profile for a human subject.

### Example 17: Identification of Drugs for the Treatment of Diseases of the Hypothalamus

To identify drugs for the treatment of hypothalamus disorders, such as obesity, inflammation, infertility and aging, hypothalamus stem cells obtained from a human subject as described above are plated in 96-well plates. Each well is contacted with a candidate drug and a biological property selected from cell viability, proliferation, apoptosis, stem-ness, and differentiation is measured by one or more techniques including XTT, MTT, BrdU, EdU, WST-1, luminescent ATP, Ki67, CFSE, trypan blue, Live/Dead, Annexin V, mitochondrial membrane potential, cytochrome C, JC-1 dye, biotracker mitochondria dye, mitochondrial apoptosis detection, mitochondrial depolarization stress damage, Caspase-3 dye, Caspase-3,7, Caspase-8, Caspase-9, Caspase activity, TUNEL, DNA fragmentation detection, cell death detection, H2A.X phosphorylation, immuno-fluorescent staining, neurosphere formation, and self-renewal.

### Example 18: Scaling Up Hypothalamus Stem Cell Production

Pluripotent cells are grown into aggregates in suspension and then differentiated into hypothalamus stem cells as described below.

High quality human pluripotent stem cell lines are cultured in 2D and passaged using EDTA in phosphate buffered saline onto a non-tissue culture treated vessel devoid of matrix proteins in basal media supplemented with 0.12 ng/ml transforming growth factor β (rh TGFβ) and 4 ng/ml fibroblast growth factor (rh bFGF), 2 mM L-glutamine, 0.1 mM beta-mercaptoethanol, 1% non-essential amino acid stock, and, optionally, with 1,000 to 3,000μ/ml rh LIF, 25 ng/ml IL-6, 25 ng/ml sIL-6R, and 15% knock-out serum replacement. Initial cultures are supplemented with Y-27632 to promote survival. The vessel is placed in a CO₂ incubator at 37° C. with rotation at 30-100 rotations per minute.

The pluripotent stem cell lines are expanded, and the cultures are passaged when the density of the cells reaches 1-2×10⁶ viable cells/ml. several times.

To induce differentiation, the media is replaced with differentiation basal media supplemented with glutamine, insulin, 1×B27, 1 μM LDN193189 and 10 μM SB431542, and after 48 hours the media is further supplemented with 1 μM SAG, 10 μM IWR and 1 μM PMN from day 2 to day 9, with several passages. After 9 days of culture, the media is replaced with differentiation basal media supplemented with glutamine, 1×N2, 1×B27, insulin, 10 μM DAPT, and 0.01 μM retinoic acid.

Cell cultures are harvested between day 9 and day 15 post-differentiation induction.

### Example 19: Isolation of Hypothalamus Stem Cell Exosomes from Biological Fluids

Samples of biological fluids, including cerebral spinal fluid, serum, plasma, amniotic fluid, nasal swab, are obtained from a subject and diluted with phosphate buffered saline. The samples are then treated with beads coated with antibodies that recognize hypothalamus stem cell exosomes, including antibodies to SLC18A2, SLC18A3, SLC17A6, SHISAL2B, BRS3, DLK-1, GABRE, GOLT1A, TMEM114, ABCG2, CD133, CXCR4, FGF R4, Frizzled-9, Glut1, SSEA-1 Notch-1 and Notch-2.

The antibody-coated exosomes are washed with phosphate buffered saline containing exosome-depleted serum and detergent to prevent non-specific binding, and the exosomes conjugated with the antibodies are isolated and purified.

The exosomes thus obtained are combined with biomarkers for prognostics and diagnostic determinations, such as prenatal diagnostics, or engineered for targeted therapy or research as described above.

### Example 20: Treatment of Chronic Inflammation by Delivery of Hypothalamus Stem Cell Exosomes Loaded with NFkappaB Inhibitor

Purified exosomes produced as described above are loaded with a therapeutic that suppresses NFkappaB activation by electroporation and delivered across the blood brain barrier of a subject suffering from chronic inflammation. The therapy results in gradual decrease of chronic inflammation, until complete healing.

### Example 21: Human Hypothalamus Stem Cells

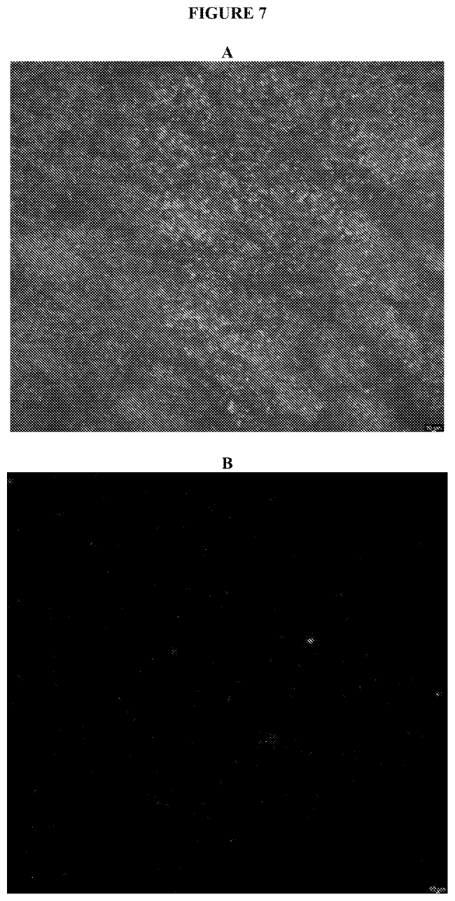

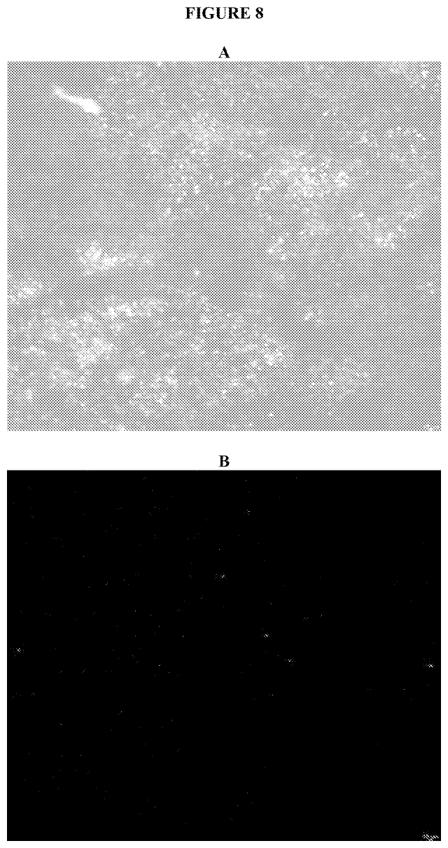

Human hypothalamus stem cells created by differentiation of induced pluripotent stem cells express markers of both hypothalamus cells (FIG. 7) and of neural stem cells (FIG. 8). BYS0112 induced pluripotent stem cells were obtained from ATCC. BYS0112 cells were cultured in mTesr Plus medium on plates pre-coated with hESC-qualified Matrigel. Matrigel was aliquoted in volumes per manufacturer recommendations for the dilution factor, and diluted in 25 ml of PBS or DMEM/F12 without serum. Tissue culture plates were treated with dilute Matrigel for at least one hour using a volume of 1 ml/well of a 6-well plate, or 6 ml/100 mm dish. BYS0112 cells were passaged as aggregates using enzyme-free dissociation methods based on EDTA.

To initiate differentiation, BYS0112 cells were dissociated enzymatically, seeded in a tissue culture-treated vessel at a density of 70,000 cells/cm² and grown overnight in mTesr Plus medium containing the ROCK inhibitor Y-27632. The next day media was changed to differentiation media A. Cells were cultured in this media for 2 days (48 hours), with daily media changes. Cells were then cultured in differentiation media B for a time period between 4 and 7 days with daily media changes. Cells were then cultured in differentiation media C until hypothalamus stem cells were produced. Hypothalamus stem cells were observed between day 9 of culture and day 14 of culture and exosomes were collected from cell culture supernatant during this time. Once pluripotent are differentiated into hypothalamus stem cells, hypothalamus stem cells are found in culture for several days, weeks and in some cases months under self-renewal conditions. In the absence of growth factors that promote the differentiation of hypothalamus stem cells, the hypothalamus stem cells can be maintained as hypothalamus stem cells in culture indefinitely in differentiation media C, and immortalized by exogenously expressing an immortalizing oncogene such as hTERT or SV40 largeT antigen.

The composition of differentiation media A is basal media supplemented with 1× B27 plus, 1× glutamax, d-glucose, insulin 4 ug/ml, super-oxide dismutase 2 ug/ml, 1 uM LDN193189 and 10 uM SB431542. The media is optionally supplemented with recombinant sonic hedgehog protein, 10 uM IWR-1endo, 1 uM smoothened agonist SAG or 1 uM purmorphamine (PMN).

The composition of differentiation media B is basal media supplemented with 1× B27 plus, 1× glutamax, d-glucose, insulin 4 ug/ml, super-oxide dismutase 2 ug/ml, 10 uM IWR-1endo, 1 uM purmorphamine (PMN) and 1 uM SAG. In some experiments the amount of B27 was reduced to 0.5× and N₂ supplement was added at a concentration of 0.5×.

The composition of differentiation media C is basal media supplemented with 1× B27 plus, 1× glutamax, d-glucose, insulin 4 ug/ml, super-oxide dismutase 2 ug/ml, and 10 uM DAPT. In some cases the amount of B27 plus was reduced to 0.5× and N₂ supplement was added at a concentration of 0.5×. In other cases 1×B27 plus was replaced with 1×N₂.

### Example 22: Human Hypothalamus Stem Cell Exosome Purification and Characterization

Hypothalamus stem cell exosomes were purified from hypothalamus stem cell conditioned medium using ultrafiltration followed by size exclusion separation. Standard laboratory protection equipment (gloves, coat, goggles, and mask) were used on all steps of sample preparation and analysis to prevent samples contamination with dust particles. Conditioned cell culture media from hypothalamus stem cells was collected after 48 hours of culture. The media was frozen until further analysis. The conditioned medium was thawed on ice on the day of analysis. For protease inhibitor (PI) preparation, 1 tablet of protease inhibitor was sufficient for 10 ml of media. 10× stock solution of protease inhibitors (PhosStop and Complete PI cocktail, 1 tablet each) was prepared by dissolving 2 tablets of each in 2 ml of 0.22 μm of filtered PBS. Then, 1.5 ml of PI solution was added to 15 ml of thawed conditioned cell culture media.

Ultrafiltration was performed to remove proteins and low molecular compounds and to concentrate the sample for loading on the size exclusion column. Conditioned medium with protease inhibitors was quickly centrifuged at 1000×g to remove large debris and cell fragments. The ultrafiltration devices containing 100 kDa molecular weight cut-off (MWCO) PES membrane were rinsed with PBS before use by adding 1 ml of PBS to the filter and spinning the device at 3,000 xg for 5 min. Any remaining retentate, as well as the eluate, were discarded. Conditioned medium was loaded into pre-rinsed filter device and centrifuged at 3000 g for 30 minutes until the retentate volume reached 100 μl. The retentate was transferred to new tubes. Membranes were washed with 0.2 ml of PBS by pipetting up and down at least 10 times. The wash was repeated two times. The wash solutions were combined with conditioned medium retentate to a final volume 0.5 ml.

Size Exclusion Separation was then performed. Size exclusion separation resolves particles by size. Larger particles elute first on the column, followed by proteins and small molecular weight compounds. Izon 35 nm qEV original column was washed with 20 ml of freshly filtered PBS. With cap closed, the buffer from the top of the column was removed and 500 μl of the sample was loaded. Cap was opened immediately and 0.5 ml fractions were collected. The column was not allowed to dry out at any time, and fresh PBS was added at the top when needed to maintain the flow. First 6 fractions (3 ml)—void volume, were discarded. Exosome fractions 7, 8 and 9 were collected and pooled together. The exosome fractions were concentrated by centrifugal filter devices. A total of 50 μl was recovered after centrifugation and transferred into a new tube. The filter membranes were rinsed with 100 μl of PBS by pipetting up and down 10 times. The wash buffer was combined with the exosome retentate and washed one more time. The total volume of the exosome fraction was 250 μL. Protein concentration was measured.

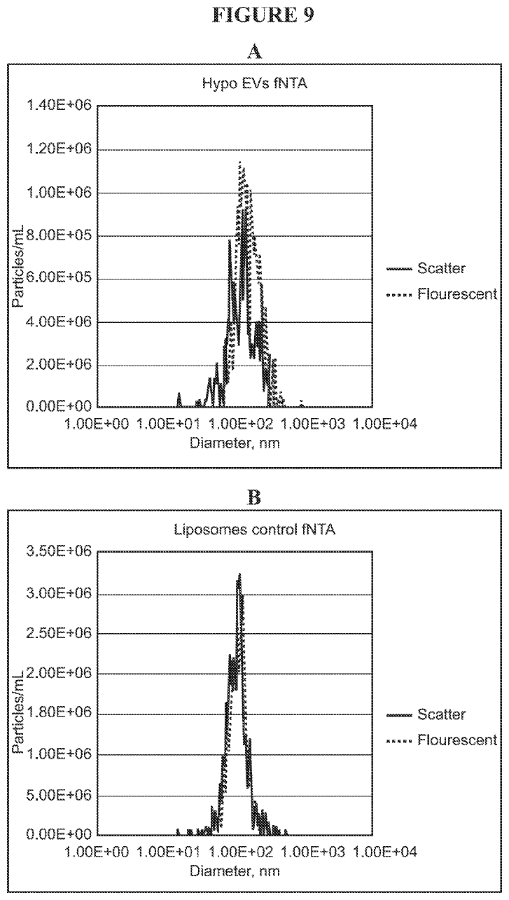

Nanoparticle tracking analysis (NTA) was used to quantify the number of hypothalamus stem cell exosomes and to determine their size (FIG. 9). NTA is a technique for visualization and analysis of dilute aqueous particles solutions. Particles are visualized as they scatter light of the laser beam passing through the sample cell. Particles with size under 1000 nm freely move in solution under Brownian motion. Visualized particles tracks are recorded by camera. Track length traveled by particles per unit of time is analyzed by software and allows determination of a size, size distribution profile and concentration of particles with a diameter of approximately 30-1000 nm. Particle size is calculated to a sphere equivalent hydrodynamic radius through the Stokes-Einstein equation.

Fluorescent NTA technique involves labeling of intact exosomal membrane with a fluorescent dye and then performing the analysis in scatter and fluorescent modes. This technique allows exclusion of contaminant particles, such as protein aggregates, lipoproteins, etc. and assess purity of exosome samples. The analysis shown in FIG. 9 was performed by fluorescent NTA equipped with 520 nm laser, 550 nm long pass cut off filter and sCMOS camera. DI water was filtered on the day of analysis through 0.22 m syringe filter and its purity confirmed by NTA prior to the study. Instrument calibration was performed with 100 nm polystyrene fluorescent beads. For exosome labeling, 12 μl of reaction buffer were mixed with 2 μl of dye and 5 μl of sample. The mixture was vortexed for 15 seconds and samples were incubated at RT for 10 minutes. Liposomes were used as labeling control: 1 μl of liposomes was mixed with 12 μl of reaction buffer and 2 μl of dye. Dilutions were made by mixing DI water filtered through 0.2 μm syringe filter with corresponding volumes of a sample.

### Example 23: Visualization of Human Hypothalamus Stem Cell Exosomes by Electron Microscopy

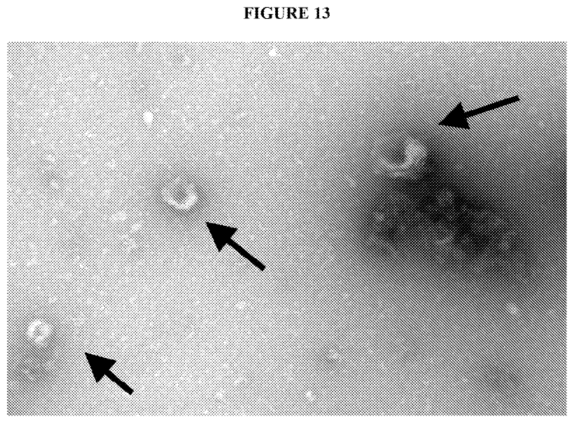

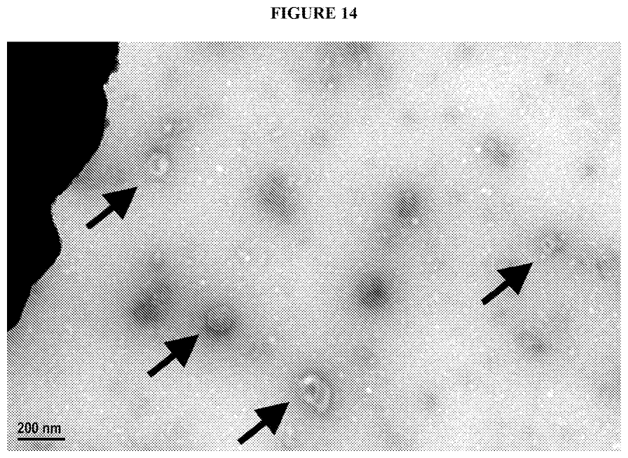

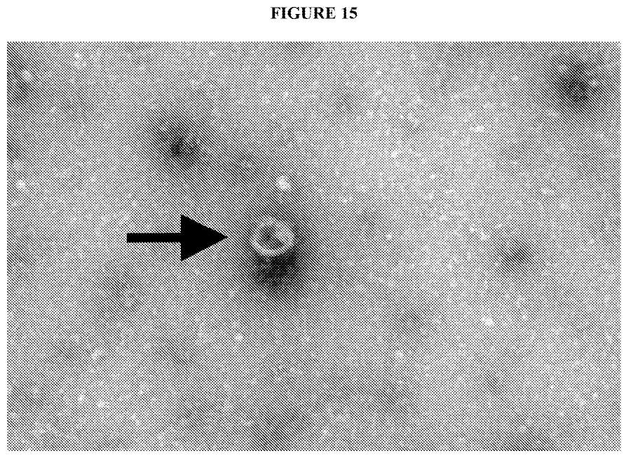

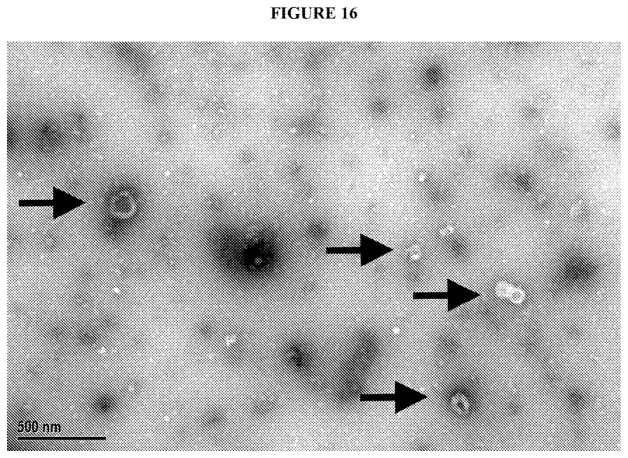

Transmission electron microscopy was used to visualize the human hypothalamus stem cell exosomes (FIGS. 13-16). Copper carbon grids were glow-discharged immediately prior to loading the samples. Sample was processed undiluted. Grid was floated on 10 μl sample drop for 15 minutes, washed twice with water by floating on the drop of water for 30 seconds, and negatively stained with 2% uranyl acetate by floating on the drop of stain for 30 seconds. The grid was blot dried with Whatman paper and imaged by electron microscope.

### Example 24: Capillary Western Blots Confirm that Human Hypothalamus Stem Cell Exosomes Express Proteins Associated with Exosomes

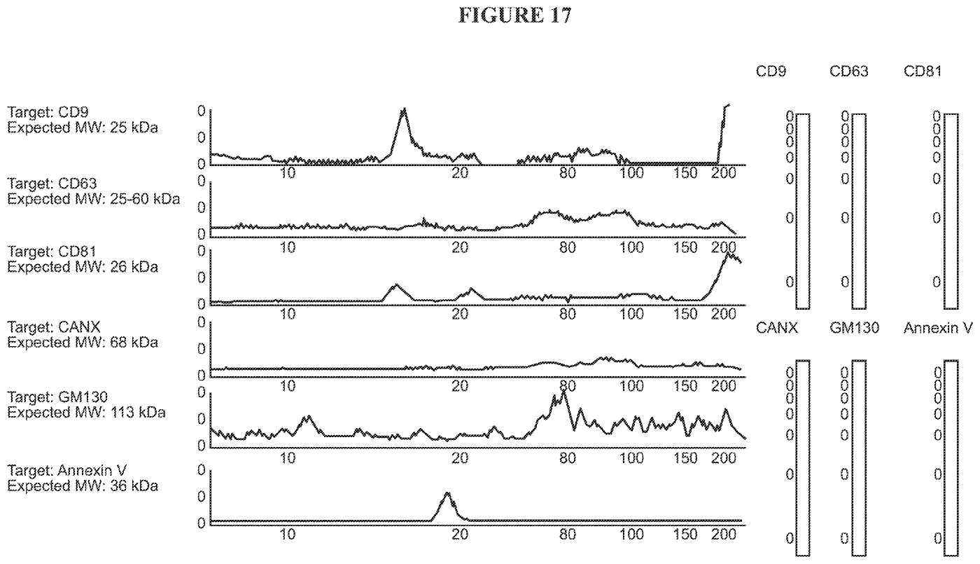

Purified hypothalamus stem cell exosomes were analyzed for expression of 4 positive controls—CD9, CD63, CD81 and Annexin V—and 2 negative controls—GM130 and CANX—by capillary western blot (FIG. 17). All four positive controls showed corresponding peaks. GM130 was not detected in the sample, while some levels of CANX were observed. Samples and reagents were loaded into an assay plate and then the samples were loaded into the capillary automatically and separated by size as they migrated through a stacking and separation matrix. The separated proteins were then immobilized on the capillary wall by proprietary, photoactivated capture chemistry. Target proteins are identified using a primary antibody and immunoprobed using an HRP-conjugated secondary antibody and chemiluminescent substrate. The resulting chemiluminescent signal is detected and quantitated.

### Example 25: Hypothalamus Stem Cell Exosomes Decrease Inflammation in Human Cells

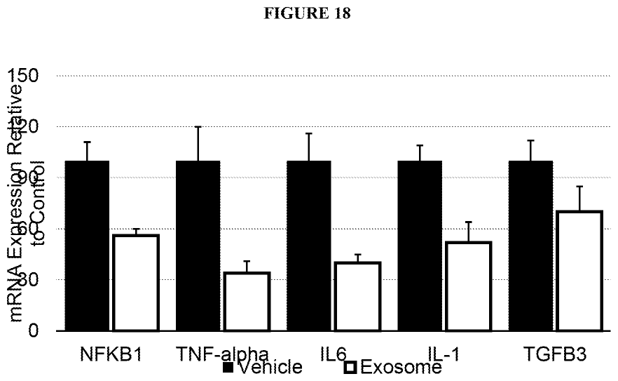

Inflammation in the human brain has been associated with neurodegenerative disease. To validate the utility of human hypothalamus stem cell exosomes the human astrocyte cell line (CHLA-03-AA cells) was treated with inflammatory cytokines to simulate inflammation and then treated with hypothalamus stem cells or vehicle control. Gene expression of genes associated with inflammation was then assessed. CHLA-03-AA cells were grown in DMEM:F12 Medium with 20 ng/ml human recombinant EGF, 20 ng/ml human recombinant basic FGF, and B-27 Supplement to a final concentration of 2% (v/v). CHLA-03-AA cells were seeded at 50,000 cells/cm² into the wells of the 24-well carrier plates (1.9 cm²/well). 10 ng/ml recombinant human IL-1β and 10 ng/ml recombinant human TNF-α was added to stimulate inflammation. Mock-exosomes isolated from a no-cell negative control or 50 ug of hypothalamus stem cell exosomes was added to the cultures. RNA was purified and 1 ug of RNA was used for reverse transcription. The ability of human hypothalamus stem cell exosomes to abate transcription of inflammatory genes was measured by qPCR using the following primers: TNF-alpha FWD: CTCTTCTGCCTGCTGCACTTTG, RVS: ATGGGCTACAGGCTTGTCACTC; Interleukin-6 FWD: GACAGCCACTCACCTCTTCAG; RVS: TTCTGCCAGTGCCTCTTTGCTG; Interleukin-lbeta FWD: CCACAGACCTTCCAGGAGAATG, RVS: GTGCAGTTCAGTGATCGTACAGG; NFkappaB1 FWD: GCAGCACTACTTCTTGACCACC, RVS: TCTGCTCCTGAGCATTGACGTC; TGFB3 FWD: CTAAGCGGAATGAGCAGAGGATC, RVS: TCTCAACAGCCACTCACGCACA; Beta actin FWD: CACCATTGGCAATGAGCGGTTC, RVS: AGGTCTTTGCGGATGTCCACGT. FIG. 18 shows that human hypothalamus stem cell exosomes are able to reduce the expression of inflammatory genes in human cells.

### Example 26: Hormones Can Be Loaded Onto Hypothalamus Stem Cell Exosomes

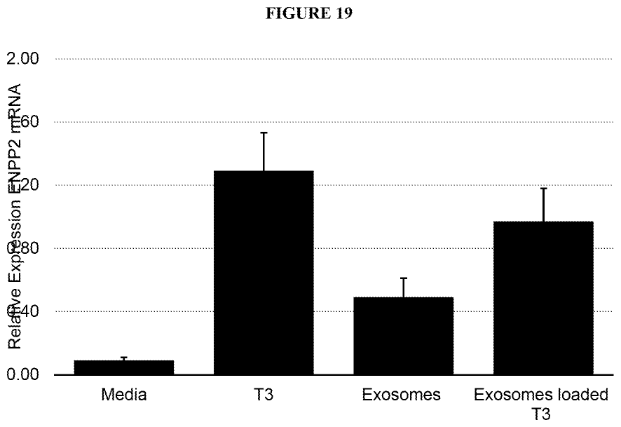

The thyroid hormone receptor ligand T3 was loaded into hypothalamus stem cell exosomes that were then administered to human cells to determine whether loading of bioactive molecules into hypothalamus stem cells causes biological effects. The gene ectonucleotide pyrophosphatase/phosphodiesterase 2 (ENPP2) is a gene that is activated upon activation of the thyroid-hormone receptor by the ligand T3. FIG. 19 shows that transcription of the gene ENPP2 is activated following administration of exosomes loaded with T3, similar to treatment with T3.

Human SK-N-AS neuroblastoma cells were obtained from ATCC and grown in DMEM supplemented with 0.1 mM Non-Essential Amino Acids (NEAA) and 10% exosome-depleted fetal bovine serum that was charcoal-stripped the night prior to incubator with T3. Human hypothalamus stem cells were loaded with T3 by sonication using six cycles of 30 s on/off for a total of 3 min with 2 min cooling period in a dismembrator with a 0.25″ tip at 20% amplitude. Exosomes were allowed to recover for 60 minutes by incubation at 37° C. Unincorporated siRNA was removed by purifying exosomes using an exosome spin column (MW 3000). Some exosomes are loaded by electroporation at a voltage of 750 with 10 pulses of 20 ms, or by passive loading by incubation of exosome and drugs in a 1:1 ratio.

SK-N-AS cells were treated either with media only mock-exosome negative control, 100 nM T3, 50 ug hypothalamus stem cell exosomes, or 50 ug hypothalamus stem cell exosomes loaded with T3. Cells were incubated for 24 hours before RNA purification. ug of RNA was used for reverse transcription. The ability to activate the thyroid hormone receptor was measured by qPCR. The hENPP2 gene was amplified using the primer sequences FWD: ACTCCGTGAAGGCAAAGAGA, and RVS: CAAGATCCGGAGATGTTGGT. The housekeeping gene cyclophilin A was amplified with the primer sequences FWD: GGCAAATGCTGGACCCAACAC and RVS: TGCCATTCCTGGACCCAAAGC.

### Example 27: siRNA is Loaded into Hypothalamus Stem Cell Exosomes

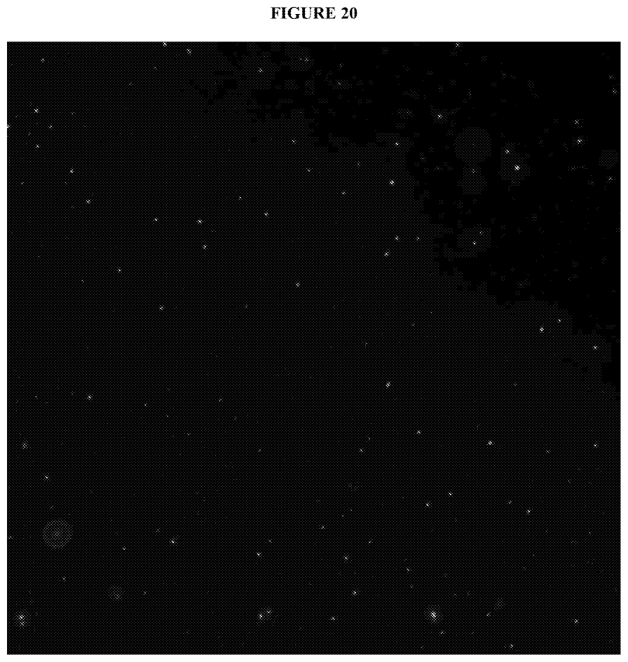

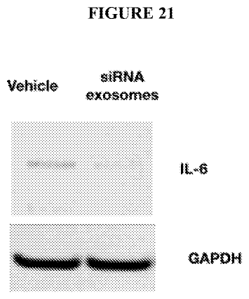

Human hypothalamus stem cells were engineered to express exogenously added siRNA molecules. Human hypothalamus stem cells were loaded with control siRNA or siRNA molecules to IL-6 by electroporation at a voltage of 750V with 10 pulses of 20 ms. Electroporation cuvettes were pre-chilled on ice for 30 minutes prior to electroporation. 10 ug of exosomes in PBS were mixed with 0.47 ug of the respective siRNA in a total volume of 150 ul with citric acid buffer. Following electroporation exosomes were incubated at 37° C. for 30 minutes to allow recovery of the exosomal membrane. Unincorporated siRNA was removed by purifying exosomes using an exosome spin column (MW 3000). FIG. 20 shows siRNA incorporation as indicated by fluorescence of hypothalamus stem cell exosomes. 50 ug of siRNA loaded exosomes or vehicle control was then administer to CHLA-03-AA cells treated with 10 ng/ml recombinant human IL-1β and 10 ng/ml recombinant human TNF-α was added to stimulate inflammation. Cells were lysed in RIPA buffer and protein expression of IL-6 was measured by Western Blotting. FIG. 21 shows silencing of IL-6 following treatment with hypothalamus stem cell exosomes loaded with IL-6 siRNA.

In addition to electroporation, siRNA incorporation in hypothalamus stem cell exosomes can be obtained by direct loading of siRNA into the exosomes ex vivo, transfection, fusion of lipids and by transfection of cargo-loading chaperone proteins.

### Example 28: Blood Brain Barrier

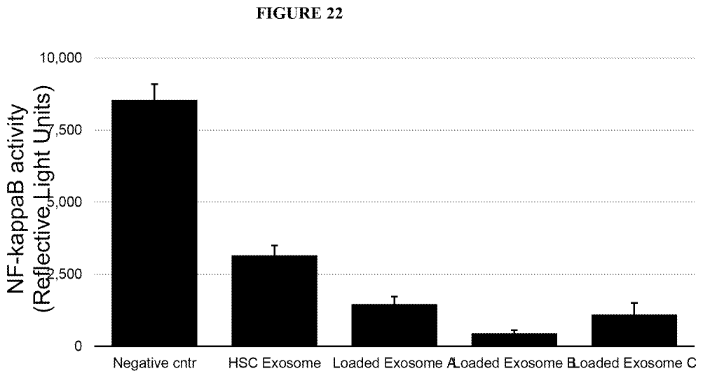

An in vitro model was used in FIG. 22 to show the ability of NFκB inhibitors to cross the blood-brain barrier following loading onto hypothalamus stem cell exosomes. HBEC-5i human cerebral endothelial cells were seeded at 50,000 cells/cm² on 0.1% gelatin coated, 24-well, 0.4 μm-pore, translucent membrane inserts (0.3 cm²/insert) to establish a polarized monolayer representative of the BBB. Cells were cultured in DMEM:F12 medium supplemented with 40 μg/ml endothelial growth factor, 10% exosome-depleted fetal bovine serum, and 1% penicillin/streptomycin solution. Human astrocytes were grown in DMEM:F12 Medium with 20 ng/ml human recombinant EGF, 20 ng/ml human recombinant basic FGF, and B-27 Supplement to a final concentration of 2% (v/v). CHLA-03-AA cells were seeded at 50,000 cells/cm² into the wells of the 24-well carrier plates (1.9 cm2/well). Cells were cultured in a humidified 37° C. incubator with 5% CO² until endothelial cells reached confluence. At confluence, endothelial cell inserts were co-cultured with CHLA-03-AA in DMEM:F12 Medium with 20 ng/ml human recombinant EGF, 20 ng/ml human recombinant basic FGF, and B-27 Supplement to a final concentration of 2%. 10 ng/ml recombinant human IL-1β and 10 ng/ml recombinant human TNF-α was added to the culture along with mock-exosomes isolated from a no-cell negative control, or 50 ug of the following: human hypothalamus stem cell exosomes, human hypothalamus stem cell exosomes loaded with NFKB inhibitor II-CAS 749886-87-1 (loaded exosome A), human hypothalamus stem cell exosomes loaded with NFKB inhibitor III-CAS 380623-76-7 (loaded exosome B), human hypothalamus stem cell exosomes loaded with NFKB inhibitor IV-CAS 139141-12-1 (loaded exosome C) for 24 hours. Hypothalamus stem cell exosomes were loaded with various NFKB inhibitors by incubation of exosomes with 200 uM of the respective inhibitor in a 1:1 v/v ratio for 1 hour at room temperature. After loading, the exosomes were separated from unincorporated drug using an exosome spin column (MW 3000). NFKB activation was assessed 24 hours after incubation by measuring activated nuclear NFκB protein.

Transfer of proteins across the blood brain barrier can be achieved by loading hypothalamus stem cell exosomes with proteins by transient or stable transfection, which is ideal for cytoplasmic proteins, or by using a vector that creates a chimeric protein that contains an exosome-anchoring protein such as HPV-E7 or HIV-1 Nef.

Hypothalamus stem cell exosomes can also be loaded ex vivo with proteins by sonication, freeze-thaw cycles, treatment with saponin or extrusion. For saponin treatment, exosomes are diluted to 0.15 mg/ml, the protein is diluted in PBS (0.5 mg/ml) abd added to 250 μl of exosomes to a final concentration of 0.1 mg/ml total protein. The protein mixed with exosomes is supplemented with 0.2% saponin and placed on an orbital shaker for 20 min at RT. For freeze-thaw cycles, the protein solution is added to exosomes as described above, incubated for 30 min, then rapidly freezed at −80° C., and thawed at RT. The freeze-thaw cycle is repeated three times. For sonication, the protein is mixed with exosomes and sonicated (500 v, 2 kHz, 20% power, 6 cycles by 4 sec pulse/2 sec pause), cooled down on ice for 2 min, and then sonicated again. For extrusion, protein mixed with exosomes is extruded (×10 times) through an extruder with 200 nm-pore diameter. Exosomes loaded with protein are purified from free protein by gel-filtration chromatography or by column. While these methods are useful for proteins, they may also be used for nucleic acids, hormones and small molecule drugs.

It should be recognized that illustrated embodiments are only examples of the disclosed product and methods and should not be considered a limitation on the scope of the invention. Rather, the scope of the invention is defined by the following claims. We therefore claim as our invention all that comes within the scope and spirit of these claims.

1. A method for preventing, treating, managing or controlling a disease or disorder associated with hypothalamus malfunction in a subject in need thereof, wherein the method comprises administering to the subject a pharmaceutical composition comprising a therapeutically effective amount of purified human hypothalamus exosomes, and wherein the purified human hypothalamus exosomes are engineered to express one or more foreign molecules, or are loaded with a biologically active compound.

2. The method of claim 1, wherein the purified human hypothalamus exosomes are produced by (i) culturing induced pluripotent stem cell in culture media and enabling reproducible differentiation of induced pluripotent stem cells into cells of ventral diencephalon hypothalamic cell lineage; (ii) identifying hypothalamic cells between day 0 and day 15 of cell culture that display hypothalamus markers and neuronal stem cell markers; (iii) isolating hypothalamus stem cells at hypothalamus marker and neuronal stem cell marker maximal expression; (iv) purifying isolated human hypothalamus stem cells; (v) isolating and purifying exosomes from the culture media; and (vi) analyzing purified exosomes by one or more of western blot analysis, flow cytometry, nanoparticle-tracking analysis, density-gradient ultracentrifugation, pull-down assay, and real-time PCR.

3. The method of claim 2, wherein the neuronal stem cell markers are one or more of Sox2⁺, Bmi-1⁺, nestin⁺, Musashi1⁺ and Cxcr4⁺, and wherein the hypothalamus markers comprise one or more of NK2 homeobox 1 (Nkx2.1) and homeobox protein orthopedia.

4. The method of claim 3, wherein the purified human hypothalamus stem cells are Rax^{hi}, Sox1^{lo}, Sox2⁺ and Bmi-1⁺.

5. The method of claim 4, wherein hypothalamus marker and neuronal stem cell marker maximal expression is between day 7 and day 15 of cell culture.

6. The method of claim 5, wherein the purified human hypothalamus stem cells are analyzed for immunophenotype, neurosphere formation and ability to give rise to mature hypothalamus neurons.

7. The method of claim 6, wherein the ability to give rise to mature hypothalamus neurons is measured by detecting expression of one or more neuropeptide markers.

8. The method of claim 7, wherein the one or more neuropeptide markers comprise Otp, Rax, neuropeptide Y (NPY), cocaine amphetamine regulated transcript (CART), α-melanocyte stimulating hormone (α-MSH), neuropeptide Y receptor Y2 (NPYR), ghrelin receptor (GhrR), and melanin concentrating hormone (MCH).

9. The method of claim 1, wherein the one or more foreign molecules comprise one or more of a growth factor, a nucleic acid, a cytokine, a vaccine, a inactivated viral protein, a drug, a chemotherapeutic and a biologically active molecule.

10. The method of claim 9, where the nucleic acid is DNA, messenger RNA, micro RNA, or small interfering RNA.

11. The method of claim 9, wherein the growth factor is human growth hormone (hGH), platelet-derived growth factor-BB (PDGF-BB), or bone morphogenetic protein (BMP).

12. The method of claim 9, wherein the cytokine is an interleukin, an interferon, or a synthetic cytokine.

13. The method of claim 1, wherein the biologically active compound is one or more of a corticosteroid receptor agonist, an inhibitor of TNF-alpha, an inhibitor of NF-kB, an inhibitor of PI3K, a small molecule inhibitor of cytokine signaling, a small molecule antagonist of cIAP1/2 and XIAP (X-linked inhibitor of apoptosis protein), a mitochondria-derived activator of caspase (SMAC) mimetic; AT406, an inhibitor of IKK Complex, an inhibitor of Nuclear Factor Kappa-B Kinase Subunit Gamma (IKKγ) inhibitors, an IKKε and Tank Binding Kinase 1 (TBK1) inhibitor, a cytokine inhibitor, a hormone receptor agonist, a hormone receptor antagonist, NF-κB Inducing Kinase (NIK) inhibitor, a nuclear receptor agonist, a nuclear receptor antagonist, an inhibitor of ubiquitin-proteasome system, a proteasome inhibitor, NAE (NEDD8 activating enzyme) inhibitor, a deubiquitination (DUB) inhibitor, a molecule inhibiting nuclear translocation, a DNA binding and transcriptional activator of NF-κB, polyphenols-like curcumin, capsaicin, apigenin, oleandrin, quercetin, resveratrol, cinnamaldehyde, and epigallocatechin-3-gallate.

14. The method of claim 1, wherein the disease or disorder is one or more of an infection, a viral disease, a degenerative disease, an autoimmune disease, a genetic disorder, a dermatological disorder, a topical inflammation, a metabolic syndrome, a cancer, or a damaged tissue in need of repair.

15. The method of claim 1, wherein the disorder is tissue damage from a degenerative or ischemic disease, and wherein the disease is dementia, neurodegenerative disease, high blood pressure, heart disease, cognitive decline from aging, Alzheimer's disease, Parkinson's disease, stroke, epilepsy, migraine, multiple sclerosis, neuropathy, spinal cord injury, traumatic brain injury, hearing loss, eye blindness, alcoholism, alcohol withdrawal, alcoholic neuropathy, neuropathic pain, multiple sclerosis, amyotrophic lateral sclerosis, Huntington's disease, ischemic brain injury, musculoskeletal trauma, over-use injury, age-related degeneration, cartilage tissue degeneration, arthritis, osteoarthritis, degenerative joint disease, rheumatoid arthritis, psoriatic arthritis, infectious septic arthritis, osteoporosis, a fracture, bone fracture, vertebral compression fracture, bone remodeling, dermal disorder, wound, prolonged inflammation, free radical damage, apoptosis, necrosis, coronary artery disease, myocardial infarction, blinding eye disease, age-related macular degeneration, metabolic syndrome, diabetes, polycystic ovary syndrome, fatty liver disease, cholesterol gallstones, asthma, sleep disturbance, or cancer.

16. The method of claim 15, wherein the pharmaceutical composition is in form of pill, capsule, tablet, gel, bead, lozenge, dragee, granule, aerogel, crumble, snap, liquid, powder, nebulizer, infusion, cream, lotion, depot, food product or wound healing composition.

17. The method of claim 16, wherein the pharmaceutical composition is administered by intracranial, oral, parenteral, topical, mucosal, sub-mucosal, pulmonary, nasal, or transdermal administration.

18. The method of claim 17, wherein the pharmaceutical composition is administered in a daily therapeutically effective dosage of at least 0.01 mg, 0.1 mg, 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 100 mg, or 200 mg of engineered or loaded exosomes/kg of the subject.

19. The method of claim 18, wherein the pharmaceutical composition is administered to the subject at least 1, 2, 3, or 4 weeks prior to the disease or disorder onset, within 24 after the disease or disorder onset, or at least 1, 2, 3, or 4 weeks after the disease or disorder onset, in single or multiple doses.

20. The method of claim 19, wherein the pharmaceutical composition is co-administered with one or more bioactive agents, and wherein the one or more bioactive agents are one or more of an antiviral agent, an anti-inflammatory agent, a hormone, a chemotherapeutic, a neural agent, or a pro-drug.


SEQUENCE LISTING (16 sequences)

SEQ ID NO: 1 - DNA, 22 bp, artificial sequence
  synthetic construct
ctcttctgcctgctgcactttg

SEQ ID NO: 2 - DNA, 22 bp, artificial sequence
  synthetic construct
atgggctacaggcttgtcactc

SEQ ID NO: 3 - DNA, 21 bp, artificial sequence
  synthetic construct
gacagccactcacctcttcag

SEQ ID NO: 4 - DNA, 22 bp, artificial sequence
  synthetic construct
ttctgccagtgcctctttgctg

SEQ ID NO: 5 - DNA, 22 bp, artificial sequence
  synthetic construct
ccacagaccttccaggagaatg

SEQ ID NO: 6 - DNA, 23 bp, artificial sequence
  synthetic construct
gtgcagttcagtgatcgtacagg

SEQ ID NO: 7 - DNA, 22 bp, artificial sequence
  synthetic construct
gcagcactacttcttgaccacc

SEQ ID NO: 8 - DNA, 22 bp, artificial sequence
  synthetic construct
tctgctcctgagcattgacgtc

SEQ ID NO: 9 - DNA, 23 bp, artificial sequence
  synthetic construct
ctaagcggaatgagcagaggatc

SEQ ID NO: 10 - DNA, 22 bp, artificial sequence
  synthetic construct
tctcaacagccactcacgcaca

SEQ ID NO: 11 - DNA, 22 bp, artificial sequence
  synthetic construct
caccattggcaatgagcggttc

SEQ ID NO: 12 - DNA, 22 bp, artificial sequence
  synthetic construct
aggtctttgcggatgtccacgt

SEQ ID NO: 13 - DNA, 20 bp, artificial sequence
  synthetic construct
actccgtgaaggcaaagaga

SEQ ID NO: 14 - DNA, 20 bp, artificial sequence
  synthetic construct
caagatccggagatgttggt

SEQ ID NO: 15 - DNA, 21 bp, artificial sequence
  synthetic construct
ggcaaatgctggacccaacac

SEQ ID NO: 16 - DNA, 21 bp, artificial sequence
  synthetic construct
tgccattcctggacccaaagc